(19) United States
(12) Patent Application Publication
(10) Pub. No.: US 2014/0303163 A1
(43) Pub. Date: Oct. 9, 2014

(54) USE OF KINASE INHIBITORS IN PREVENTING AND TREATING INFLAMMATORY DISORDER

(71) Applicant: Ying Luo, Shanghai (CN)

(72) Inventor: Ying Luo, Shanghai (CN)

(21) Appl. No.: 14/007,130

(22) PCT Filed: Mar. 24, 2011
(86) PCT No.: PCT/CN2011/072096

§ 371 Date: Oct. 25, 2013

(51) Int. Cl. C07D 471/04; C07D 403/04

(52) U.S. Cl. CPC C07D 471/04; C07D 403/04
USPC 514/234.5; 544/297; 514/275; 544/122; 544/295; 514/252.18

(57) ABSTRACT

The present invention discloses use of compounds of formula (I) in preventing and treating inflammatory disorder.

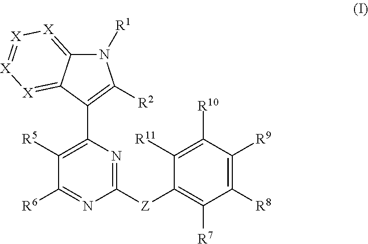

### FIELD OF THE INVENTION

Described herein are methods of using compounds to treat, or prevent disorders or conditions associated with one or more protein kinases

### BACKGROUND OF THE INVENTION

Inflammatory disorders are associated with abnormal cellular responses triggered by protein kinase-mediated events. Some CDK inhibitors have effect of overriding pro-inflammatory signalling and driving neutrophil apoptosis. These inhibitors include, but are not limited to Roscovitine. R-roscovitine could enhance resolution of carrageenan-induced pleurisy. And R-rescovitine also had effect on resolution of bleomycin-induced lung inflammation and serum-induced arthristis. (Rossi et al, Nat. Med. (2006) 12:9 1056-1064) The cyclin-dependent kinase inhibitor R-roscovitine down-regulated Mcl-1 to override pro-inflammatory signalling and drive neutrophil apoptosis. (Andrew E. Leitch, et al, Eur. J. Immunol 2010 40: 1127-1138) CDK9 promotes RNA synthesis in genetic programmes for cell growth, differentiation and viral pathogenesis. CDK9 inhibition contributes to the anticancer activity of most CDK inhibitors under clinic investigation. CDK9 inhibitors might become specific antiretroviral agents, particularly as they might prevent drug resistance. But it is still lack of selective inhibitors in clinical development, which means a need of offering a more effective agent for preventing and treating inflammatory disorders is an exigent task in the art.

### SUMMARY OF THE INVENTION

Presented herein are methods, for preventing and treating inflammatory disorders.

In one aspect is a use of a compound or pharmaceutically acceptable salt, solvate, or prodrug thereof, having the structure of Formula (I), for preparing pharmaceutical agents of treating or preventing inflammatory disorder:

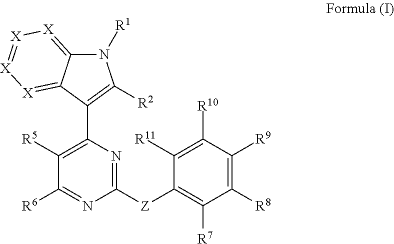

wherein each X is —C(R³)— or —N—, provided that at least two X are —C(R³)—;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl;
R¹, R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²; L^{A} is a covalent bond or an alkyl group; L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—; R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃; the inflammatory disorder is selected from ARDS, Sepsis, or Rheumatoid Arthritic.

In some embodiments, Z is —NR^{a}—. In one aspect, R^{a} is H.

In one aspect, R⁵ and R⁶ are each H.

In some embodiments, R² is H.

In some embodiments, each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃.

In some embodiments, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²; L^{A} is a covalent bond or an alkyl group; L^{B} is a covalent bond, —O—, —NR³²—, —NH—, —C(═O)O—, —C(═O)NH—, or —SO₂NH—; R³² is H, substituted or unsubstituted alkyl, haloalkyl, substituted or unsubstituted aryl, substituted or unsubstituted heteroaryl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

In one aspect, the compound having the structure of Formula (Ia):

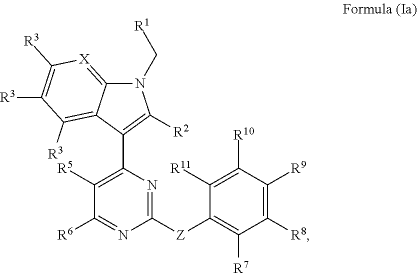

wherein:
X is —C(R³)— or —N—;
R¹ is unsubstituted C₁-C₆ alkyl, unsubstituted or substituted aryl, or unsubstituted or substituted heteroaryl;
R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —SO₂NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl.

In one aspect, X is N. In another aspect, X is —C(R³)—.

In some embodiments, Z is —NR^{a}—.

In some embodiments, R¹ is unsubstituted C₁-C₆ alkyl or unsubstituted or substituted phenyl. In some other embodiments, R¹ is unsubstituted C₁-C₆ alkyl.

In some embodiments, each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃.

In some embodiments, R², R⁵ and R⁶ are each H.

In other embodiments, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²; L^{A} is a covalent bond or an alkyl group; L^{B} is a covalent bond, —O—, —NR³²—, —NH—, —CO(═O)—, —C(═O)NR³²—, —C(═O)NH—, —SO₂NR³²—, or —SO₂NH—; R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

In one aspect, the compound having the structure of Formula (Ib):

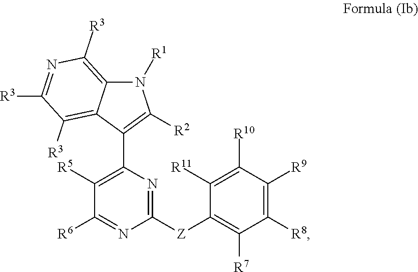

wherein:
R¹, R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl.

In some embodiments, Z is —NR^{a}—.

In some embodiments, R¹ is unsubstituted C₁-C₆ alkyl.

In some embodiments, each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃.

In other embodiments, R², R⁵ and R⁶ are each H.

In some embodiments, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³², L^{A} is a covalent bond or an alkyl group; L^{B} is a covalent bond, —O—, —NR³²—, —NH—, —C(═O)O—, —C(═O)NR³²—, —C(═O)NH—, —SO₂NR³²—, or —SO₂NH—; R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

In one aspect, the compound having the structure of Formula (Ic):

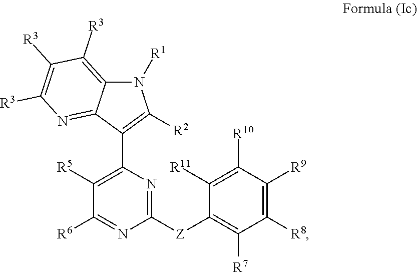

wherein:
R¹, R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;

R^{a} is H or alkyl.

In some embodiments, Z is —NR^{a}—.

In some embodiments, R¹ is unsubstituted C₁-C₆ alkyl.

In some embodiments, each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃.

In some embodiments, R², R⁵ and R⁶ are each H.

In some other embodiments, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³², L^{A} is a covalent bond or an alkyl group; L^{B} is a covalent bond, —O—, —NR³²—, —NH—, —CO(═O)—, —C(═O)NR³²—, —C(═O)NH—, —SO₂NR³²—, or —SO₂NH—; R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

In some embodiments, L^{A} is a covalent bond.

In some embodiments, L^{B} is a covalent bond, —O—, —NH—, —C(═O)O—, —C(═O)NH—, or —SO₂NH—.

In some embodiments, R³² is H, substituted or unsubstituted alkyl, or haloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃. In some embodiments, R³² is H, substituted or unsubstituted alkyl, or haloalkyl.

In one aspect, R¹, R³, R⁷, R⁸, R⁹, R¹⁰, R¹¹ are as defined in Table 1, Table 2, Table 3 and Table 4. In one aspect, R², R⁵, and R⁶ are each H and R¹, R³, R⁷, R⁸, R⁹, R¹⁰, R¹¹ are as defined in Table 1, Table 2, Table 3 and Table 4.

Any combination of the groups described above for the various variables is contemplated herein.

In one aspect, provided is a method for treating or preventing inflammatory disorders by administrating to the patient an effective amount of a compound, or pharmaceutically acceptable salt, solvate, or prodrug thereof, the compound having the structure of Formula (I), preferably the compound having the structure of Formula (Ia), (Ib), or (Ic). The inflammatory disorder is selected from ARDS, Sepsis, or Rheumatoid Arthritic.

In one aspect, provided is a method for inhibiting undesired cyclin-dependent kinase 9 activity in a cell comprising contacting the cell with a compound of Formula (I).

In one aspect, provided is a method for treating inflammatory disorder in a patient, in which the inflammatory disorder is characterized by undesired cyclin-dependent kinase 9 activity, comprising administering to the patient an amount of a compound of Formula (I) effective for inhibiting the undesired cyclin-dependent kinase 9 activity. In some embodiments, the inflammatory disorder is selected from ARDS, Sepsis, or Rheumatoid Arthritic.

In any of the aforementioned aspects are further embodiments that include single administrations of the effective amount of the compound of Formula (I), including further embodiments in which: (i) the compound of Formula (I) is administered once; (ii) the compound of Formula (I) is administered to the mammal multiple times over the span of one day; (iii) the compound of Formula (I) is administered continually; or (iv) the compound of Formula (I) is administered continuously.

In any of the aforementioned aspects are further embodiments that include multiple administrations of the effective amount of the compound of Formula (I), including further embodiments in which (i) the compound of Formula (I) is administered in a single dose; (ii) the time between multiple administrations is every 6 hours; (iii) the compound of Formula (I) is administered to the mammal every 8 hours. In further or alternative embodiments, the method comprises a drug holiday, wherein the administration of the compound of Formula (I) is temporarily suspended or the dose of the compound of Formula (I) being administered is temporarily reduced; at the end of the drug holiday, dosing of the compound of Formula (I) is resumed. The length of the drug holiday can vary from 2 days to 1 year.

In one aspect, compounds of Formula (I) described herein are administered to a human. In some embodiments, compounds of Formula (I) described herein are orally administered.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DESCRIPTION OF FIGURES

FIG. 1 showed that compound 18 induced cell apoptosis with dose-dependent manner

FIG. 2 showed that compound 18 inhibited CDK9's activity on Pol II phosphorylation.

FIG. 3 showed time diagram of rabbit neutrophil apoptosis, including which FIG. 3A showed the cells at the beginning of isolation, FIG. 3B showed the cells at 24 hours after isolation, FIG. 3C showed the cells at 48 hours after isolation, and FIG. 3D showed the percentage of neutophil apoptosis vs the time after isolation.

FIG. 4 showed neutophil apoptosis induced by different compounds (×40), including which FIG. 4A showed the effect of negative control, FIG. 4B showed the effect of LPS (lipopolysaccharide) (1 μg/mL), FIG. 4C showed the effect of LPS (5 μg/mL), FIG. 4D showed the effect of LPS (10 μg/mL), FIG. 4E showed the effect of compound 18 (0.5 μM), FIG. 4F showed the effect of compound 18 (2 μM), FIG. 4G showed the effect of compound 18 (10 μM), and FIG. 4H showed the effect of Roscovitine (20 μM).

FIG. 5 showed comparison of the effect of compound 18 with that of other compounds in inducing neutrophil apoptosis, including which FIG. 5A showed the result of negative control, FIG. 5B showed the result of LPS (1 μg/mL), FIG. 5C showed the result of LPS (5 μg/mL), FIG. 5D showed the result of LPS (10 μg/mL), FIG. 5E showed the result of compound 18 (0.5 μM), FIG. 5F showed the result of compound 18 (2 μM), FIG. 5G showed the result of compound 18 (10 μM), FIG. 5H showed the result of Roscovitine (20 μM), and FIG. 5I showed the apoptosis percentage vs various compounds.

### DETAILED DESCRIPTION OF THE INVENTION

Cyclin-dependent kinases (CDKs) are serine/threonine protein kinases that associate with various cyclin subunits, playing pivotal roles in the regulation of cell cycle progression and transcriptional cycle. Ten distinct CDKs (CDK1-9 and 11) are involved in a variety of important regulatory pathways in eukaryotic cells, including cell-cycle control, apoptosis, neuronal physiology, differentiation and transcription.

In one aspect, CDKs are classified into two major groups, reflecting their functions. The cell cycle regulator CDKs composed primarily of CDK1, CDK2, CDK3, CDK4 and CDK6 function with their cyclin partners including cyclin A, B, D1, D2, D3, E, and F to regulate promotion of the cell cycle. The transcription regulator CDKs, which include CDK7, CDK8, CDK9 and CDK11 work together with cyclin C, H, K, L1, L2, T1 and T2, tend to play roles in transcriptional regulation.

CDKs also play a role in apoptosis and T-cell development, which is predominantly due to the CDK functions in regulation of transcription.

Described herein are compounds which are detailed described in PCT/CN2009/075214. The contents of the PCT application are included in the application.

### Compounds

Compounds of Formula (I) include, but are not limited to, those described in Table 1:

TABLE 1

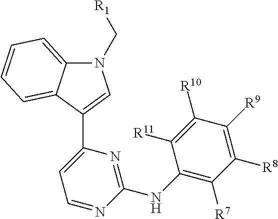

| Com- | | | | | | |
|---|---|---|---|---|---|---|
| pound | | | | | | |
| no. | R¹ | R⁷ | R⁸ | R⁹ | R¹⁰ | R¹¹ |
| 1 | —H | H | —OH | H | H | H |
| 2 | —H | H | H | —OH | H | H |
| 3 | —H | —Cl | H | —OH | H | H |
| 4 | —H | —CH₃ | H | —OH | H | H |
| 5 | —H | —CH₃ | H | —OH | H | —CH₃ |
| 6 | —CH₃ | H | H | —OH | H | H |
| 7 | —CH₃ | H | —OH | H | H | H |
| 8 | —CH₃ | —CH₃ | H | —OH | H | H |
| 9 | —CH₃ | —CH₃ | H | —OH | H | —CH₃ |
| 10 | -Phenyl | H | H | —OH | H | H |
| 11 | -Phenyl | H | —OH | H | H | H |

Compound in Table 1 are named:
3-(4-(1-Methyl-1H-indol-3-yl)pyrimidin-2-ylamino)phenol (Compound 1)
4-(4-(1-Methyl-1H-indol-3-yl)pyrimidin-2-ylamino)phenol (Compound 2)
3-Chloro-4-(4-(1-methyl-1H-indol-3-yl)pyrimidin-2-ylamino)phenol (Compound 3)
3-Methyl-4-[4-(1-methyl-1H-indol-3-yl)-pyrimidin-2-ylamino]-phenol (Compound 4)
3,5-Dimethyl-4-[4-(1-methyl-1H-indol-3-yl)-pyrimidin-2-ylamino]-phenol (Compound 5)
4-[4-(1-Ethyl-1H-indol-3-yl)-pyrimidin-2-ylamino]-phenol (Compound 6)
3-[4-(1-Ethyl-1H-indol-3-yl)-pyrimidin-2-ylamino]-phenol (Compound 7)
4-[4-(1-Ethyl-1H-indol-3-yl)-pyrimidin-2-ylamino]-3-methyl-phenol (Compound 8)
4-[4-(1-Ethyl-1H-indol-3-yl)-pyrimidin-2-ylamino]-3,5-dimethyl-phenol (Compound 9)
4-[4-(1-Benzyl-1H-indol-3-yl)-pyrimidin-2-ylamino]-phenol (Compound 10)
3-[4-(1-Benzyl-1H-indol-3-yl)-pyrimidin-2-ylamino]-phenol (Compound 11)

In one aspect, compounds of Formula (I) include, but are not limited to, those described in Table 2:

TABLE 2

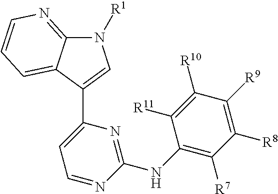

| Compound | | | | | | |
|---|---|---|---|---|---|---|
| no. | R¹ | R⁷ | R⁸ | R⁹ | R¹⁰ | R¹¹ |
| 12 | —CH₂CH₃ | H | H | —OH | H | H |
| 13 | —CH₂CH₃ | H | —OH | H | H | H |
| 14 | —CH₂CH₃ | H | —NO₂ | H | H | H |
| 15 | —CH₂CH₂CH₃ | H | H | —OH | H | H |
| 16 | CH₂CH₂CH₃ | H | —OH | H | H | H |
| 17 | CH₂CH₂CH₃ | H | —NO₂ | H | H | H |
| 18 | CH₂CH₃ | H | —SO₂NH₂ | H | H | H |
| 19 | CH₂CH₃ | H | —CONH₂ | H | H | H |
| 20 | CH₂CH₃ | H | CN | H | H | H |
| 21 | CH₂CH₃ | H | H | —SO₂NH₂ | H | H |
| 22 | CH₂CH₂CH₃ | H | —SO₂NH₂ | H | H | H |
| 23 | CH₂CH₂CH₃ | H | H | SO₂NH₂ | H | H |
| 24 | CH₂CH₂CH₃ | H | -sulfonylmorpholine | Me | H | H |
| 25 | CH₂CH₂CH₃ | H | -sulfonylpiperazine | H | H | H |
| 26 | CH₂CH₂CH₃ | H | H | -sulfonylpiperazine | H | H |
| 27 | CH₂CH₂CH₃ | H | H | -sulfonylmorpholine | H | H |
| 28 | CH₂CH₂CH₃ | H | -sulfonyl-4-methylpiperazine | H | H | H |
| 29 | CH₂CH₂CH₃ | H | H | -sulfonyl-4-methylpiperazine | H | H |
| 30 | CH₂CH₂CH₃ | H | -morpholine | H | H | H |
| 31 | CH₂CH₂CH₃ | H | H | -morpholine | H | H |
| 32 | CH₂CH₂CH₃ | H | -piperazine | | H | H |
| 33 | CH₂CH₂CH₃ | H | H | -piperazine | H | H |
| 34 | CH₂CH₂CH₃ | H | —C(═O)-4-methylpiperazine | H | H | H |
| 35 | CH₂CH₂CH₃ | H | | -4-methylpiperazine | H | H |
| 36 | CH₂CH₂CH₃ | H | —C(═O)-(4-acetyl piperazin-1-yl)- | H | H | H |
| 37 | CH₂CH₂CH₃ | H | H | —C(═O)-(4-acetyl piperazin-1-yl)- | H | H |

Compound in Table 2 are named:
4-[4-(1-Ethyl-1H-pyrrolo[2,3-b]pyridin-3-yl)-pyrimidin-2-ylamino]-phenol (Compound 12)
3-[4-(1-Ethyl-1H-pyrrolo[2,3-b]pyridin-3-yl)-pyrimidin-2-ylamino]-phenol (Compound 13)
[4-(1-Ethyl-1H-pyrrolo[2,3-b]pyridin-3-yl)-pyrimidin-2-yl]-(3-nitro-phenyl)-amine (Compound 14)
4-[4-(1-Propyl-1H-pyrrolo[2,3-b]pyridin-3-yl)-pyrimidin-2-ylamino]-phenol (Compound 15)
3-[4-(1-Propyl-1H-pyrrolo[2,3-b]pyridin-3-yl)-pyrimidin-2-ylamino]-phenol (Compound 16)
(3-Nitro-phenyl)-[4-(1-propyl-1H-pyrrolo[2,3-b]pyridin-3-yl)-pyrimidin-2-yl]-amine (Compound 17)
3-(4-(1-Ethyl-1H-pyrrolo[2,3-b]pyridin-3-yl)pyrimidin-2-ylamino)benzenesulfonamide (Compound 18)
3-(4-(1-Ethyl-1H-pyrrolo[2,3-b]pyridin-3-yl)pyrimidin-2-ylamino)benzamide (Compound 19).
3-(4-(1-ethyl-1H-pyrrolo[2,3-b]pyridin-3-yl)pyrimidin-2-ylamino)benzonitrile (Compound 20).
4-(4-(1-ethyl-1H-pyrrolo[2,3-b]pyridin-3-yl)pyrimidin-2-ylamino)benzenesulfonamide (Compound 21).
3-(4-(1-propyl-1H-pyrrolo[2,3-b]pyridin-3-yl)pyrimidin-2-ylamino)benzenesulfonamide (Compound 22).
4-(4-(1-Propyl-1H-pyrrolo[2,3-b]pyridin-3-yl)pyrimidin-2-ylamino)benzenesulfonamide (Compound 23).
N-(4-Methyl-3-(morpholinosulfonyl)phenyl)-4-(1-propyl-1H-pyrrolo[2,3-b]pyridin-3-yl)pyrimidin-2-amine
(Compound 24).
N-(3-(Piperazin-1-ylsulfonyl)phenyl)-4-(1-propyl-1H-pyrrolo[2,3-b]pyridin-3-yl)pyrimidin-2-amine (Compound 25).
N-(4-(Piperazin-1-ylsulfonyl)phenyl)-4-(1-propyl-1H-pyrrolo[2,3-b]pyridin-3-yl)pyrimidin-2-amine (Compound 26).
N-(4-(Morpholinosulfonyl)phenyl)-4-(1-propyl-1H-pyrrolo[2,3-b]pyridin-3-yl)pyrimidin-2-amine (Compound 27).
N-(3-(4-Methylpiperazin-1-ylsulfonyl)phenyl)-4-(1-propyl-1H-pyrrolo[2,3-b]pyridin-3-yl)pyrimidin-2-amine
(Compound 28).
N-(4-(4-Methylpiperazin-1-ylsulfonyl)phenyl)-4-(1-propyl-1H-pyrrolo[2,3-b]pyridin-3-yl)pyrimidin-2-amine
(Compound 29).
N-(3-Morpholinophenyl)-4-(1-propyl-1H-pyrrolo[2,3-b]pyridin-3-yl)pyrimidin-2-amine (Compound 30).
N-(4-Morpholinophenyl)-4-(1-propyl-1H-pyrrolo[2,3-b]pyridin-3-yl)pyrimidin-2-amine (Compound 31).
N-(3-(Piperazin-1-yl)phenyl)-4-(1-propyl-1H-pyrrolo[2,3-b]pyridin-3-yl)pyrimidin-2-amine (Compound 32).
N-(4-(Piperazin-1-yl)phenyl)-4-(1-propyl-1H-pyrrolo[2,3-b]pyridin-3-yl)pyrimidin-2-amine (Compound 33).
(4-Methylpiperazin-1-yl)(3-(4-(1-propyl-1H-pyrrolo[2,3-b]pyridin-3-yl)pyrimidin-2-ylamino)phenyl)methanone
(Compound 34).
N-(4-(4-Methylpiperazin-1-yl)phenyl)-4-(1-propyl-1H-pyrrolo[2,3-b]pyridin-3-yl)pyrimidin-2-amine (Compound 35).
1-(4-(3-(4-(1-Propyl-1H-pyrrolo[2,3-b]pyridin-3-yl)pyrimidin-2-ylamino)phenyl)piperazin-1-yl)ethanone (Compound
36).
1-(4-(4-(4-(1-Propyl-1H-pyrrolo[2,3-b]pyridin-3-yl)pyrimidin-2-ylamino)phenyl)piperazin-1-yl)ethanone (Compound
37).

In one aspect, compounds of Formula (I) include, but are not limited to, those described in Table 3:

TABLE 3

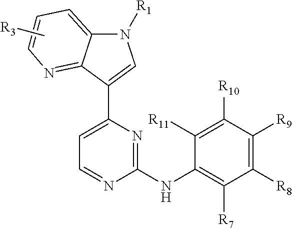

| Compound | | | | | | | |
|---|---|---|---|---|---|---|---|
| no. | R¹ | R³ | R⁷ | R⁸ | R⁹ | R¹⁰ | R¹¹ |
| 38 | CH₂CH₂H₃ | 5-Cl, 7-CH₃ | H | H | —OH | H | H |
| 39 | CH₂CH₂H₃ | 5-Cl, 7-CH₃ | H | H | —SO₂NH₂ | H | H |
| 40 | CH₂CH₂H₃ | 5-Cl, 7-CH₃ | H | —OH | H | H | H |
| 41 | CH₂CH₂H₃ | 5-Cl, 7-CH₃ | Me | H | —OH | H | H |
| 42 | CH₂CH₂H₃ | 5-Cl, 7-CH₃ | H | —SO₂NH₂ | H | H | H |
| 43 | CH₂CH₂H₃ | 5-Cl, 7-CH₃ | H | -sulfonylmorpholine | Me | H | H |
| 44 | CH₂CH₂H₃ | 5-Cl, 7-CH₃ | H | -sulfonylpiperazine | H | H | H |
| 45 | CH₂CH₂H₃ | 5-Cl, 7-CH₃ | H | H | -sulfonylpiperazine | H | H |
| 46 | CH₂CH₂H₃ | 5-Cl, 7-CH₃ | H | -piperazine | H | H | H |
| 47 | CH₂CH₂H₃ | 5-Cl, 7-CH₃ | H | H | -piperazine | H | H |
| 48 | CH₂CH₂H₃ | 5-Cl, 7-CH₃ | H | -morpholine | H | H | H |
| 49 | CH₂CH₂H₃ | 5-Cl, 7-CH₃ | H | H | -morpholine | H | H |
| 50 | CH₂CH₂H₃ | 5-Cl, 7-CH₃ | H | —C(═O)-(4-acetyl piperazin-1-yl)- | H | H | H |
| 51 | CH₂CH₂H₃ | 5-Cl, 7-CH₃ | H | H | —C(═O)-(4-acetyl piperazin-1-yl)- | H | H |
| 52 | CH₂CH₂H₃ | 5-Cl, 7-CH₃ | H | C(═O)-morpholine | H | H | H |
| 53 | CH₂CH₂H₃ | 5-Cl, 7-CH₃ | H | H | C(═O)-morpholine | H | H |
| 54 | CH₂CH₂H₃ | 7-CH₃ | H | —SO₂NH₂ | H | H | H |
| 55 | CH₂CH₂H₃ | 7-CH₃ | H | H | —SO₂NH₂ | H | H |
| 56 | CH₂CH₂H₃ | 7-CH₃ | H | -sulfonylpiperazine | H | H | H |
| 57 | CH₂CH₂H₃ | 7-CH₃ | H | H | -sulfonylpiperazine | H | H |
| 58 | CH₂CH₂H₃ | 7-CH₃ | H | -piperazine | H | H | H |
| 59 | CH₂CH₂H₃ | 7-CH₃ | H | H | -piperazine | H | H |
| 60 | CH₂CH₂H₃ | 7-CH₃ | H | -morpholine | H | H | H |
| 61 | CH₂CH₂H₃ | 7-CH₃ | H | H | -morpholine | H | H |
| 62 | CH₂CH₂H₃ | 7-CH₃ | H | —C(═O)-(4-acetyl | H | H | H |
| | | | | piperazin-1-yl)- | | | |
| 63 | CH₂CH₂H₃ | 7-CH₃ | H | H | —C(═O)-(4-acetyl piperazin-1-yl)- | H | H |
| 64 | CH₂H₃ | 7-CH₃ | H | —SO₂NH₂ | H | H | H |
| 65 | CH₂H₃ | 7-CH₃ | H | H | —SO₂NH₂ | H | H |
| 66 | CH₂H₃ | 7-CH₃ | H | —C(═O)-morpholine | H | H | H |
| 67 | CH₂H₃ | 7-CH₃ | H | H | —C(═O)-morpholine | H | H |
| 68 | CH₂H₃ | 7-CH₃ | H | -piperazine | H | | |
| 69 | CH₂H₃ | 7-CH₃ | H | H | -piperazine | H | H |

Compounds in Table 3 are named:
4-(4-(5-Chloro-7-methyl-1-propyl-1H-pyrrolo[3,2-b]pyridin-3-yl)pyrimidin-2-ylamino)phenol (Compound 38).
4-(4-(5-Chloro-7-methyl-1-propyl-1H-pyrrolo[3,2-b]pyridin-3-yl)pyrimidin-2-ylamino)benzenesulfonamide
(Compound 39)
3-(4-(5-Chloro-7-methyl-1-propyl-1H-pyrrolo[3,2-b]pyridin-3-yl)pyrimidin-2-ylamino)phenol (Compound 40).
4-(4-(5-Chloro-7-methyl-1-propyl-1H-pyrrolo[3,2-b]pyridin-3-yl)pyrimidin-2-ylamino)-3-methylphenol (Compound
41).
3-(4-(5-Chloro-7-methyl-1-propyl-1H-pyrrolo[3,2-b]pyridin-3-yl)pyrimidin-2-ylamino)benzenesulfonamide
(Compound 42)
4-(5-Chloro-7-methyl-1-propyl-1H-pyrrolo[3,2-b]pyridin-3-yl)-N-(4-methyl-3-(morpholinosulfonyl)phenyl)pyrimidin-
2-amine (Compound 43)
4-(5-Chloro-7-methyl-1-propyl-1H-pyrrolo[3,2-b]pyridin-3-yl)-N-(3-(piperazin-1-ylsulfonyl)phenyl)pyrimidin-2-
amine (Compound 44)
4-(5-Chloro-7-methyl-1-propyl-1H-pyrrolo[3,2-b]pyridin-3-yl)-N-(4-(piperazin-1-ylsulfonyl)phenyl)pyrimidin-2-
amine (Compound 45)
4-(5-Chloro-7-methyl-1-propyl-1H-pyrrolo[3,2-b]pyridin-3-yl)-N-(3-(piperazin-1-yl)phenyl)pyrimidin-2-amine
(Compound 46).
4-(5-Chloro-7-methyl-1-propyl-1H-pyrrolo[3,2-b]pyridin-3-yl)-N-(4-(piperazin-1-yl)phenyl)pyrimidin-2-amine
(Compound 47)
4-(5-Chloro-7-methyl-1-propyl-1H-pyrrolo[3,2-b]pyridin-3-yl)-N-(3-morpholinophenyl)pyrimidin-2-amine
(Compound 48).
4-(5-Chloro-7-methyl-1-propyl-1H-pyrrolo[3,2-b]pyridin-3-yl)-N-(4-morpholinophenyl)pyrimidin-2-amine
(Compound 49).
1-(4-(3-(4-(5-Chloro-7-methyl-1-propyl-1H-pyrrolo[3,2-b]pyridin-3-yl)pyrimidin-2-ylamino)phenyl)piperazin-1-
yl)ethanone (Compound 50).
1-(4-(4-(4-(5-Chloro-7-methyl-1-propyl-1H-pyrrolo[3,2-b]pyridin-3-yl)pyrimidin-2-ylamino)phenyl)piperazin-1-
yl)ethanone (Compound 51).
(3-(4-(5-Chloro-7-methyl-1-propyl-1H-pyrrolo[3,2-b]pyridin-3-yl)pyrimidin-2-
ylamino)phenyl)(morpholino)methanone (Compound 52).
(4-(4-(5-Chloro-7-methyl-1-propyl-1H-pyrrolo[3,2-b]pyridin-3-yl)pyrimidin-2-
ylamino)phenyl)(morpholino)methanone (Compound 53).
3-(4-(7-Methyl-1-propyl-1H-pyrrolo[3,2-b]pyridin-3-yl)pyrimidin-2-ylamino)benzenesulfonamide (Compound 54).
4-(4-(7-Methyl-1-propyl-1H-pyrrolo[3,2-b]pyridin-3-yl)pyrimidin-2-ylamino)benzenesulfonamide (Compound 55).
4-(7-Methyl-1-propyl-1H-pyrrolo[3,2-b]pyridin-3-yl)-N-(3-(piperazin-1-ylsulfonyl)phenyl)pyrimidin-2-amine
(Compound 56).
4-(7-Methyl-1-propyl-1H-pyrrolo[3,2-b]pyridin-3-yl)-N-(4-(piperazin-1-ylsulfonyl)phenyl)pyrimidin-2-amine
(Compound 57).
4-(7-Methyl-1-propyl-1H-pyrrolo[3,2-b]pyridin-3-yl)-N-(3-(piperazin-1-yl)phenyl)pyrimidin-2-amine (Compound 58).
4-(7-Methyl-1-propyl-1H-pyrrolo[3,2-b]pyridin-3-yl)-N-(4-(piperazin-1-yl)phenyl)pyrimidin-2-amine (Compound 59).
4-(7-Methyl-1-propyl-1H-pyrrolo[3,2-b]pyridin-3-yl)-N-(3-morpholinophenyl)pyrimidin-2-amine (Compound 60).
4-(7-Methyl-1-propyl-1H-pyrrolo[3,2-b]pyridin-3-yl)-N-(4-morpholinophenyl)pyrimidin-2-amine (Compound 61).
1-(4-(3-(4-(7-Methyl-1-propyl-1H-pyrrolo[3,2-b]pyridin-3-yl)pyrimidin-2-ylamino)phenyl)piperazin-1-yl)ethanone
(Compound 62).
1-(4-(4-(4-(7-Methyl-1-propyl-1H-pyrrolo[3,2-b]pyridin-3-yl)pyrimidin-2-ylamino)phenyl)piperazin-1-yl)ethanone
(Compound 63).
3-(4-(1-Ethyl-7-methyl-1H-pyrrolo[3,2-b]pyridin-3-yl)pyrimidin-2-ylamino)benzenesulfonamide (Compound 64).
4-(4-(1-Ethyl-7-methyl-1H-pyrrolo[3,2-b]pyridin-3-yl)pyrimidin-2-ylamino)benzenesulfonamide (Compound 65).
(3-(4-(1-Ethyl-7-methyl-1H-pyrrolo[3,2-b]pyridin-3-yl)pyrimidin-2-ylamino)phenyl)(morpholino)methanone
(Compound 66).
(4-(4-(1-Ethyl-7-methyl-1H-pyrrolo[3,2-b]pyridin-3-yl)pyrimidin-2-ylamino)phenyl)(morpholino)methanone
(Compound 67).
4-(1-Ethyl-7-methyl-1H-pyrrolo[3,2-b]pyridin-3-yl)-N-(3-(piperazin-1-yl)phenyl)pyrimidin-2-amine (Compound 68).
4-(1-Ethyl-7-methyl-1H-pyrrolo[3,2-b]pyridin-3-yl)-N-(4-(piperazin-1-yl)phenyl)pyrimidin-2-amine (Compound 69).

TABLE 4

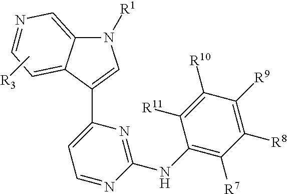

| Comp. | | | | | | | |
|---|---|---|---|---|---|---|---|
| no. | R¹ | R³ | R⁷ | R⁸ | R⁹ | R¹⁰ | R¹¹ |
| 70 | —CH₂CH₃ | 7-Cl | H | H | —OH | H | H |
| 71 | —CH₂CH₃ | 7-Cl | H | H | —SO₂NH₂ | H | H |
| 72 | —CH₂CH₃ | 7-Cl | H | —OH | H | H | H |
| 73 | —CH₂CH₃ | 7-Cl | H | —SO₂NH₂ | H | H | H |
| 74 | —CH₂CH₃ | 7-Cl | H | -sulfonylmorpholine | Me | H | H |
| 75 | —CH₂CH₃ | 7-Cl | H | —C(═O)-morpholine | H | H | H |
| 76 | —CH₂CH₃ | 7-Cl | H | H | —C(═O)-morpholine | H | H |
| 77 | —CH₂CH₃ | 7-Cl | H | -sulfonylpiperazine | H | H | H |
| 78 | —CH₂CH₃ | 7-Cl | H | H | -sulfonylpiperazine | H | H |
| 79 | —CH₂CH₃ | 7-Cl | H | -piperazine | H | H | H |
| 80 | —CH₂CH₃ | 7-Cl | H | H | -piperazine | H | H |
| 81 | —CH₂CH₃ | 7-Cl | H | -morpholine | H | H | H |
| 82 | —CH₂CH₃ | 7-Cl | H | H | -morpholine | H | H |
| 83 | —CH₂CH₃ | H | H | OH | H | H | H |
| 84 | —CH₂CH₃ | H | H | —SO₂NH₂ | H | H | H |
| 85 | —CH₂CH₃ | H | H | H | OH | H | H |
| 86 | —CH₂CH₃ | H | H | H | —SO₂NH₂ | H | H |
| 87 | —CH₂CH₃ | H | H | -sulfonylmorpholine | Me | H | H |
| 88 | —CH₂CH₃ | H | H | —C(═O)-morphline | H | H | H |
| 89 | CH₂CH₃ | H | H | H | —C(═O)-morphline | H | H |
| 90 | CH₂CH₃ | H | H | —C(═O)-(4-acetyl piperazin-1-yl)- | H | H | H |
| 91 | CH₂CH₃ | H | H | H | —C(═O)-(4-acetyl piperazin-1-yl)- | H | H |
| 92 | CH₂CH₃ | H | H | -piperazine | H | H | H |
| 93 | CH₂CH₃ | H | H | H | -piperazine | H | H |
| 94 | CH₂CH₃ | H | H | -morpholine | H | H | H |
| 95 | CH₂CH₃ | H | H | H | -morpholine | H | H |
| 96 | CH₂CH₂CH₃ | 7-Cl | H | —SO₂NH₂ | H | H | H |
| 97 | CH₂CH₂CH₃ | 7-Cl | H | H | —SO₂NH₂ | H | H |
| 98 | CH₂CH₂CH₃ | 7-Cl | H | —C(═O)-(4-acetyl piperazin-1-yl)- | H | H | H |
| 99 | CH₂CH₂CH₃ | 7-Cl | H | H | —C(═O)-(4-acetyl piperazin-1-yl)- | H | H |
| 100 | CH₂CH₂CH₃ | 7-Cl | H | H | -morpholine | H | H |
| 101 | CH₂CH₂CH₃ | 7-Cl | H | —SO₂-morpholine | Me | H | H |
| 102 | CH₂CH₂CH₃ | H | H | —SO₂-morpholine | Me | H | H |
| 103 | CH₂CH₂CH₃ | H | H | H | -morpholine | H | H |
| 104 | CH₂CH₂CH₃ | H | H | -piperazine | H | H | H |
| 105 | CH₂CH₂CH₃ | H | H | H | -piperazine | H | H |
| 106 | CH₂CH₂CH₃ | H | H | H | —C(═O)-(4-acetyl piperazin-1-yl)- | H | H |
| 107 | CH₂CH₂CH₃ | H | H | —C(═O)-piperazin- | H | H | H |
| 108 | CH₂CH₂CH₃ | H | H | —C(═O)-morpholine | H | H | H |
| 109 | CH₂CH₂CH₃ | H | H | H | —C(═O)-piperazin- | H | H |
| 110 | CH₂CH₂CH₃ | H | H | H | —C(═O)-morpholine | H | H |
| 111 | CH₂CH₂CH₃ | H | H | —SO₂NH₂ | H | H | H |
| 112 | CH₂CH₂CH₃ | H | H | H | —SO₂NH₂ | H | H |

Compounds in Table 4 are named:
4-(4-(7-Chloro-1-ethyl-1H-pyrrolo[2,3-c]pyridin-3-yl)pyrimidin-2-ylamino)phenol (Compound 70)
4-(4-(7-Chloro-1-ethyl-1H-pyrrolo[2,3-c]pyridin-3-yl)pyrimidin-2-ylamino)benzenesulfonamide (Compound 71)
3-(4-(7-Chloro-1-ethyl-1H-pyrrolo[2,3-c]pyridin-3-yl)pyrimidin-2-ylamino)phenol (Compound 72)
3-(4-(7-Chloro-1-ethyl-1H-pyrrolo[2,3-c]pyridin-3-yl)pyrimidin-2-ylamino)benzenesulfonamide (Compound 73)
4-(7-Chloro-1-ethyl-1H-pyrrolo[2,3-c]pyridin-3-yl)-N-(4-methyl-3-(morpholinosulfonyl)phenyl)pyrimidin-2-amine
(Compound 74)
(3-(4-(7-Chloro-1-ethyl-1H-pyrrolo[2,3-c]pyridin-3-yl)pyrimidin-2-ylamino)phenyl)(morpholino)methanone
(Compound 75)
(4-(4-(7-Chloro-1-ethyl-1H-pyrrolo[2,3-c]pyridin-3-yl)pyrimidin-2-ylamino)phenyl)(morpholino)methanone
(Compound 76)
4-(7-Chloro-1-ethyl-1H-pyrrolo[2,3-c]pyridin-3-yl)-N-(3-(piperazin-1-ylsulfonyl)phenyl)pyrimidin-2-amine
(Compound 77)
4-(7-Chloro-1-ethyl-1H-pyrrolo[2,3-c]pyridin-3-yl)-N-(4-(piperazin-1-ylsulfonyl)phenyl)pyrimidin-2-amine
(Compound 78).
4-(7-Chloro-1-ethyl-1H-pyrrolo[2,3-c]pyridin-3-yl)-N-(3-(piperazin-1-yl)phenyl)pyrimidin-2-amine (Compound 79).
4-(7-Chloro-1-ethyl-1H-pyrrolo[2,3-c]pyridin-3-yl)-N-(4-(piperazin-1-yl)phenyl)pyrimidin-2-amine (Compound 80).
4-(7-Chloro-1-ethyl-1H-pyrrolo[2,3-c]pyridin-3-yl)-N-(3-morpholinophenyl)pyrimidin-2-amine (Compound 81).
4-(7-Chloro-1-ethyl-1H-pyrrolo[2,3-c]pyridin-3-yl)-N-(4-morpholinophenyl)pyrimidin-2-amine (Compound 82).
3-(4-(1-Ethyl-1H-pyrrolo[2,3-c]pyridin-3-yl)pyrimidin-2-ylamino)phenol (Compound 83).
3-(4-(1-Ethyl-1H-pyrrolo[2,3-c]pyridin-3-yl)pyrimidin-2-ylamino)benzenesulfonamide (Compound 84).
4-(4-(1-Ethyl-1H-pyrrolo[2,3-c]pyridin-3-yl)pyrimidin-2-ylamino)phenol (Compound 85).
4-(4-(1-Ethyl-1H-pyrrolo[2,3-c]pyridin-3-yl)pyrimidin-2-ylamino)benzenesulfonamide (Compound 86).
4-(1-Ethyl-1H-pyrrolo[2,3-c]pyridin-3-yl)-N-(4-methyl-3-(morpholinosulfonyl)phenyl)pyrimidin-2-amine (Compound
87).
(3-(4-(1-Ethyl-1H-pyrrolo[2,3-c]pyridin-3-yl)pyrimidin-2-ylamino)phenyl)(morpholino)methanone (Compound 88).
(4-(4-(1-Ethyl-1H-pyrrolo[2,3-c]pyridin-3-yl)pyrimidin-2-ylamino)phenyl)(morpholino)methanone (Compound 89).
1-(4-(3-(4-(1-Ethyl-1H-pyrrolo[2,3-c]pyridin-3-yl)pyrimidin-2-ylamino)benzoyl)piperazin-1-yl)ethanone (Compound
90).
1-(4-(4-(4-(1-Ethyl-1H-pyrrolo[2,3-c]pyridin-3-yl)pyrimidin-2-ylamino)benzoyl)piperazin-1-yl)ethanone (Compound
91).
4-(1-Ethyl-1H-pyrrolo[2,3-c]pyridin-3-yl)-N-(3-(piperazin-1-yl)phenyl)pyrimidin-2-amine (Compound 92).
4-(1-Ethyl-1H-pyrrolo[2,3-c]pyridin-3-yl)-N-(4-(piperazin-1-yl)phenyl)pyrimidin-2-amine (Compound 93).
4-(1-Ethyl-1H-pyrrolo[2,3-c]pyridin-3-yl)-N-(3-morpholinophenyl)pyrimidin-2-amine (Compound 94).
4-(1-Ethyl-1H-pyrrolo[2,3-c]pyridin-3-yl)-N-(4-morpholinophenyl)pyrimidin-2-amine (Compound 95).
4-(4-(7-Chloro-1-propyl-1H-pyrrolo[2,3-c]pyridin-3-yl)pyrimidin-2-ylamino)benzenesulfonamide (Compound 96).
3-(4-(7-Chloro-1-propyl-1H-pyrrolo[2,3-c]pyridin-3-yl)pyrimidin-2-ylamino)benzenesulfonamide (Compound 97).
1-(4-(3-(4-(7-Chloro-1-propyl-1H-pyrrolo[2,3-c]pyridin-3-yl)pyrimidin-2-ylamino)benzoyl)piperazin-1-yl)ethanone
(Compound 98).
1-(4-(4-(4-(7-Chloro-1-propyl-1H-pyrrolo[2,3-c]pyridin-3-yl)pyrimidin-2-ylamino)benzoyl)piperazin-1-yl)ethanone
(Compound 99).
4-(7-Chloro-1-propyl-1H-pyrrolo[2,3-c]pyridin-3-yl)-N-(4-morpholinophenyl)pyrimidin-2-amine (Compound 100).
4-(7-Chloro-1-propyl-1H-pyrrolo[2,3-c]pyridin-3-yl)-N-(4-methyl-3-(morpholinosulfonyl)phenyl)pyrimidin-2-amine
(Compound 101).
N-(4-Methyl-3-(morpholinosulfonyl)phenyl)-4-(1-propyl-1H-pyrrolo[2,3-c]pyridin-3-yl)pyrimidin-2-amine
(Compound 102).
N-(4-Morpholinophenyl)-4-(1-propyl-1H-pyrrolo[2,3-c]pyridin-3-yl)pyrimidin-2-amine (Compound 103).
N-(3-(Piperazin-1-yl)phenyl)-4-(1-propyl-1H-pyrrolo[2,3-c]pyridin-3-yl)pyrimidin-2-amine (Compound 104).
N-(4-(Piperazin-1-yl)phenyl)-4-(1-propyl-1H-pyrrolo[2,3-c]pyridin-3-yl)pyrimidin-2-amine (Compound 105).
1-(4-(4-(4-(1-Propyl-1H-pyrrolo[2,3-c]pyridin-3-yl)pyrimidin-2-ylamino)benzoyl)piperazin-1-yl)ethanone (Compound
106).
Piperazin-1-yl(3-(4-(1-propyl-1H-pyrrolo[2,3-c]pyridin-3-yl)pyrimidin-2-ylamino)phenyl)methanone (Compound 107).
Morpholino(3-(4-(1-propyl-1H-pyrrolo[2,3-c]pyridin-3-yl)pyrimidin-2-ylamino)phenyl)methanone (Compound 108).
Piperazin-1-yl(4-(4-(1-propyl-1H-pyrrolo[2,3-c]pyridin-3-yl)pyrimidin-2-ylamino)phenyl)methanone (Compound 109).
Morpholino(4-(4-(1-propyl-1H-pyrrolo[2,3-c]pyridin-3-yl)pyrimidin-2-ylamino)phenyl)methanone (Compound 110).
3-(4-(1-Propyl-1H-pyrrolo[2,3-c]pyridin-3-yl)pyrimidin-2-ylamino)benzenesulfonamide (Compound 111).
4-(4-(1-Propyl-1H-pyrrolo[2,3-c]pyridin-3-yl)pyrimidin-2-ylamino)benzenesulfonamide (Compound 112).

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting.

“Compound of Formula (I)” refers to compound of Formula (I), compound of Formula (Ia), compound of Formula (Ib), compound of Formula (Ic), compound of Formula (Id).

An “alkyl” group refers to an aliphatic hydrocarbon group. The alkyl moiety may be a saturated alkyl group (which means that it does not contain any units of unsaturation, e.g. carbon-carbon double bonds or carbon-carbon triple bonds) or the alkyl moiety may be an unsaturated alkyl group (which means that it contains at least one unit of unsaturation). The alkyl moiety, whether saturated or unsaturated, may be branched, or straight chain.

The “alkyl” moiety may have 1 to 8 carbon atoms (whenever it appears herein, a numerical range such as “1 to 8” refers to each integer in the given range; e.g., “1 to 8 carbon atoms” means that the alkyl group may consist of 1 carbon atom, 2 carbon atoms, 3 carbon atoms, etc., up to and including 8 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one, two, three, four, five, or six carbon atoms in the alkyl chain Typical alkyl groups include, but are in no way limited to, methyl, ethyl, propyl, isopropyl, butyl, isobutyl, tertiary butyl, pentyl, hexyl, and the like.

The term “lower alkyl” is similarly used for groups having from 1 to 4 carbon atoms.

The term “aralkyl” is refers to alkyl-aryl, where alkyl and aryl are as defined herein.

The term “alicyclic” refers to a cyclic aliphatic group.

The term “aliphatic” takes its normal meaning in the art and includes non-aromatic groups such as alkanes, alkenes and alkynes and substituted derivatives thereof.

An “alkoxy” group refers to a (alkyl)O— group, where alkyl is as defined herein.

The term “alkylamine” refers to the N(alkyl)ₓH_{y} group, where x and y are selected from the group x=1, y=1 and x=2, y=0. In some embodiments, when x=2 and y=0, the alkyl groups taken together with the nitrogen atom to which they are attached form a cyclic ring system.

An “amide” is a chemical moiety with formula —C(═O)NHR or —NHC(═O)R, where R is selected from the group consisting of alkyl, cycloalkyl, aryl, heteroaryl (bonded through a ring carbon) and heteroalicyclic (bonded through a ring carbon). An amide may be an amino acid or a peptide molecule attached to a compound of Formula (I), thereby forming a prodrug. Any amine, or carboxyl side chain on the compounds described herein is optionally amidified, as desired. See, e.g., Greene and Wuts, Protective Groups in Organic Synthesis, 3^{rd} Ed., John Wiley & Sons, New York, N.Y., 1999, is incorporated herein by reference for such disclosure.

The term “aromatic” refers to a planar ring having a delocalized π-electron system containing 4n+2 IT electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” refers to a ring or ring system where the atoms forming the backbone of the ring are all carbon atoms. The term thus distinguishes carbocyclic from heterocyclic rings in which the ring backbone contains at least one atom which is different from carbon.

As used herein, the term “aryl” refers to an aromatic ring wherein each of the atoms forming the ring is a carbon atom. Aryl rings are formed by five, six, seven, eight, nine, or more than nine carbon atoms. Aryl groups are optionally substituted. In one aspect, an aryl is a phenyl or a naphthalenyl. Depending on the structure, an aryl group can be a monoradical or a diradical (i.e., an arylene group). In one aspect, an aryl is a C₆-C₁₀aryl.

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclohexyl, cycloheptyl, and cyclooctyl. Cycloalkyl groups may be substituted or unsubstituted.

As used herein, the term “carbohydrate derivative” refers to a compound of general formula Cₓ(H₂O)_{y} or a derivative thereof. Preferably, the carbohydrate is a mono-, di- or tri-saccharide. Monosaccharides can exist as either straight chain or ring-shaped molecules and are classified according to the number of carbon atoms they possess; trioses have three carbons, tetroses four, pentoses five and hexoses six. Each of these subgroups may be further divided into aldoses and ketoses, depending on whether the molecule contains an aldehyde group (—CHO) or a ketone group (C═O). Typical examples of monosaccharides include glucose, fructose, and galactose. Disaccharides consist of two linked monosaccharide molecules, and include for example, maltose and lactose. Trisaccharides consist of three linked monosaccharide molecules.

The term “ester” refers to a chemical moiety with formula —COOR, where R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein is esterified, if desired. Examples of procedures and specific groups to make such esters are found in sources such as Greene and Wuts, Protective Groups in Organic Synthesis, 3^{rd} Ed., John Wiley & Sons, New York, N.Y., 1999.

The term “halo” or, alternatively, “halogen” or “halide” means fluoro, chloro, bromo or iodo.

The term “haloalkyl” refers to an alkyl group in which one or more hydrogen atoms are replaced by one or more halide atoms. In one aspect, a haloalkyl is a C₁-C₆haloalkyl.

The term “fluoroalkyl” refers to a alkyl in which one or more hydrogen atoms are replaced by a fluorine atom. In one aspect, a fluoralkyl is a C₁-C₆fluoroalkyl.

The term “heteroalkyl” refers to an alkyl group in which one or more skeletal atoms of the alkyl are selected from an atom other than carbon, e.g., oxygen, nitrogen, sulfur, phosphorus or combinations thereof. In one aspect, a heteroalkyl is a C₁-C₆ heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles may be substituted with one or two oxo (═O) moieties, such as pyrrolidin-2-one.

The terms “heteroaryl” or, alternatively, “heteroaromatic” refers to an aryl group that includes one or more ring heteroatoms selected from nitrogen, oxygen and sulfur. Preferred heteroaryl groups include indole, azaindole, pyrrole, pyrazole, pyrimidine, pyrazine, pyridine, quinoline, thiophene and furan. In one aspect, a heteroaryl contains 0-3 N atoms. In another aspect, a heteroaryl contains 0-3 N atoms, 0-1 O atoms, and 0-1 S atoms. In another aspect, a heteroaryl is a monocyclic or bicyclic heteroaryl.

A “heterocycloalkyl”, “heteroalicyclic” or “non-aromatic heterocycle” refers to a cycloalkyl group that includes at least one heteroatom selected from nitrogen, oxygen and sulfur. The radicals may be fused with an aryl or heteroaryl. In some embodiments, the heterocycloalkyl is selected from oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₁₀ heterocycloalkyl. In another aspect, a heterocycloalkyl is a C₄-C₁₀ heterocycloalkyl. In one aspect, a heterocycloalkyl contains 0-2 N atoms. In another aspect, a heterocycloalkyl contains 0-2 N atoms, 0-2 O atoms or 0-1 S atoms.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “membered ring” includes any cyclic structure. The term “membered” is meant to denote the number of skeletal atoms that constitute the ring. Thus, for example, cyclohexyl, pyridinyl, pyranyl and thiopyranyl are 6-membered rings and cyclopentyl, pyrrolyl, furanyl, and thienyl are 5-membered rings.

The term “moiety” refers to a specific segment or functional group of a molecule. Chemical moieties are often recognized chemical entities embedded in or appended to a molecule.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, carbonyl, thiocarbonyl, nitro, haloalkyl, fluoroalkyl, and amino, including mono- and di-substituted amino groups, and the protected derivatives thereof. By way of example an optional substituents may be halide, —CN, —NO₂, or LₛRₛ, wherein each Lₛ is independently selected from a bond, —O—, —C(═O)—, —C(═O)O—, —S—, —S(═O)—, —S(═O)₂—, —NH—, —NHC(═O)—, —C(═O)NH—, S(═O)₂NH—, —NHS(═O)₂, —OC(═O)NH—, —NHC(═O)O—, or —(C₁-C₆ alkyl)-; and each Rₛ is selected from H, alkyl, fluoroalkyl, heteroalkyl, cycloalkyl, aryl, heteroaryl, or heterocycloalkyl. The protecting groups that may form the protective derivatives of the above substituents may be found in sources such as Greene and Wuts, above. In one aspect, optional substituents are selected from halogen, CF₃, OH, CN, NO₂, SO₃H, SO₂NH₂, SO₂Me, NH₂, COOH, CONH₂, alkoxy, —N(CH₃)₂, and alkyl.

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, the separation of stereoisomers by chiral chromatographic columns.

The methods and formulations described herein include the use of N-oxides (if appropriate), crystalline forms (also known as polymorphs), or pharmaceutically acceptable salts of compounds having the structure of Formula (I), as well as active metabolites of these compounds having the same type of activity. In some situations, compounds may exist as tautomers. All tautomers are included within the scope of the compounds presented herein. In specific embodiments, the compounds described herein exist in solvated forms with pharmaceutically acceptable solvents such as water, ethanol, and the like. In other embodiments, the compounds described herein exist in unsolvated form.

### Certain Pharmaceutical and Medical Terminology

The term “acceptable” with respect to a formulation, composition or ingredient, as used herein, means having no persistent detrimental effect on the general health of the subject being treated.

The term “kinase-dependent”, as used herein, refers to conditions or disorders that would not occur, or would not occur to the same extent, in the absence of a kinase enzyme.

The term “kinase-mediated”, as used herein, refers to refers to conditions or disorders that might occur in the absence of kinase enzyme but can occur in the presence of kinase enzyme.

The term “inflammatory disorders” refers to those diseases or conditions that are characterized by one or more of the signs of pain, heat, redness, swelling, and loss of function (temporary or permanent) Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (colitis); skin (dermatitis); or multiple organs and tissues (systemic lupus erythematosus).

The term “immunological disorders” refers to those diseases or conditions that are characterized by inappropriate or deleterious response to an endogenous or exogenous antigen that may result in cellular dysfunction or destruction and consequently dysfunction or destruction of an organ or tissue and which may or may not be accompanied by signs or symptoms of inflammation.

The term “ARDS,’ as used herein, refers to a disease or condition of the lung called Acute Respiratory Distress Syndrome, which is a fulminant lung condition in which trauma to the lungs leads to inflammation of the lungs, accumulation of fluid in the alveolar air sacs, low blood oxygen, and respiratory distress.

The term “Sepsis,” as used herein refers to a potentially serious medical condition that is characterized by a whole-body inflammatory state and the presence of a known or suspected infection.

The term “Rheumatoid Arthritis,” as used herein refers to a chronic, systemic inflammatory disorder that may affect many tissues and organs, but principally attacks synovial joints.

The terms “co-administration” or the like, as used herein, are meant to encompass administration of the selected therapeutic agents to a single patient, and are intended to include treatment regimens in which the agents are administered by the same or different route of administration or at the same or different time.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals and non-mammals Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment, the mammal is a human.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

### Therapeutic Use

In one aspect, the compounds of Formula (I) are used in the treatment of a disease, disorder, or condition mediated by one or more protein kinase selected from a CDK (such as CDK1, CDK2, CDK4, CDK5, CDK6, CDK7, CDK8, CDK9, CDK11, or other protein kinases), aurora kinase, GSK, PLK and one of Tyrosine kinases. In one aspect, the compounds of Formula (I) inhibit such protein kinases, preferably CDK9.

In one aspect, the compound of Formula (I) inhibits a CDK. In one aspect, the compound of Formula (I) inhibits a CDK selected from CDK1, CDK2, CDK7 and CDK9 at sub-micromolar IC₅₀ values, more preferably at IC₅₀ of less than 0.5 micromolar, more preferably less than 0.25 micromolar. In one aspect, the compound of Formula (I) inhibits CDK9 at IC₅₀ of less than 0.1 micromolar or less than 0.05 micromolar.

In some embodiments, the compound of Formula (I) demonstrates an antiproliferative effect in human cell lines, as measured by a standard 72 h MTT cytotoxicity assay. In some embodiments, the compound of Formula (I) exhibits an IC₅₀ value of less than 1 micromolar.

In a further aspect, there is provided a method of treating an inflammatory disease, said method comprising administering to a subject in need thereof, a compound of Formula (I) as hereinbefore defined in an effective amount. The use of a compound of the invention in the manufacture of a medicament as hereinbefore defined includes the use of the compound directly, or in any stage of the manufacture of such a medicament, or in vitro in a screening programme to identify further agents for the prevention or treatment of the hereinbefore defined diseases or conditions. A further aspect relates to the use of a compound of Formula (I) or a pharmaceutically acceptable salt or solvate or physiologically hydrolysable, solubilising or immobilising derivative thereof, in an assay for identifying candidate compounds capable of treating one or more disorders or diseases as hereinbefore defined. Preferably a compound is of use in identifying candidate compounds capable of inhibiting a protein kinase, more preferably CDK9.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### EXAMPLES

These examples are provided for illustrative purposes only and not to limit the scope of the claims provided herein.

### Example 1

### CDK9 Inhibitory Activity

### Kinase Assays.

The compounds were tested for their ability to inhibit kinases Kinase activity of representative compounds is presented in Table I.

TABLE I
Kinase inhibitory activities:
F200 = SG45 = Compound 18 in Table 2; SG48 = Compound 72 in Table 4;
SG49 = Compound 70 in Table 4; SG50 = Compound 73 in Table 4;
SG51 = Compound 111 in Table 4; SG52 = Compound 102 in Table 4;
SG53 = Compound 42 in Table 3; SG 54 = Compound 39 in Table 3;

| Compound | | F200 | SG48 | SG49 | SG50 | SG51 | SG52 | SG53 | SG54 |
|---|---|---|---|---|---|---|---|---|---|
| Target | CDK1 | 624 | 90 | 190 | 180 | 0.4 | 70 | 6840 | >10000 |
| biochemical | CDK2 | 3 | 4 | 16 | 4 | 0.5 | 30 | >10000 | 1670 |
| activity IC50 | CDK5 | 271 | 100 | 350 | 190 | 0.5 | 25 | >10000 | >10000 |
| (nM) | CDK7 | 146 | 3280 | >10000 | >10000 | 43 | 1950 | >10000 | >10000 |
| | CDK9 | 13 | 79 | 95 | 24 | 0.6 | 0.4 | 41 | 57 |
| | Aurora A | 30 | 800 | 2460 | 1280 | 50 | 1650 | 98 | 620 |
| | Aurora B | 700 | 930 | >10000 | >10000 | 59 | 1000 | 190 | 320 |
| | SRC | 1050 | >10000 | 4560 | >10000 | 200 | 3930 | >10000 | >10000 |
| | TSSK1 | >10000 | 140 | 1490 | 520 | 1.6 | >10000 | >10000 | >10000 |
| Cellular effect | HeLa | 1.7 | 3.31 | 2.69 | 0.23 | <0.1 | 0.49 | 9.93 | 3.3 |
| in mammalian | MCF-7 | 0.71 | 9.4 | >10 | 0.45 | <0.1 | 1.3 | >10 | >10 |
| cells | H460 | 1.27 | 4.66 | 2.95 | 0.315 | <0.1 | 0.22 | 2.00 | 1.03 |
| IC50 (mM)) | Jurkat | 1.31 | 1.92 | 0.61 | 0.4 | 0.005 | 0.69 | 0.36 | 1.12 |
| | THP-1 | 5.3 | >10 | 3.68 | >10 | ND | 0.41 | 1.04 | 2.95 |
| | K562 | 6.69 | 4.75 | 3.52 | 0.61 | 0.03 | 1.44 | >10 | 4.72 |
| | Molt-4 | 1.05 | ND | ND | ND | ND | ND | ND | ND |
| | HCT116 | 2.1 | 4.67 | 4.28 | 0.28 | 0.004 | 0.28 | 3.56 | 3.46 |
| | HepG2 | 0.68 | 6.1 | 5.55 | 0.41 | 0.03 | 0.57 | 1.86 | 1.26 |
| | HT29 | 0.59 | 8.31 | 8.59 | 1.16 | 0.02 | 0.45 | 0.25 | 0.15 |
| | MRC-5 | ND | >10 | 5.82 | 3.36 | <0.1 | 1.63 | >10 | 3.97 |
| | L02 | 4.3 | 8.1 | 7.34 | >10 | 0.02 | 1.40 | >10 | 8.37 |

ND—Not Determined

### Example 2

### Compound 18 (F200 or SG45) Induces Cell Apoptosis Assay

MCF-7 cells were treated with compound 18 (F200) and Roscovitine for 48 hours. Then the cells were analyzed by TUNEL assay and Western blot to detect apoptosis.

For TUNEL assay, cells were fixed and blocking in 6-well plate. DNA strand breaks were labeled by terminal deoxynucleotidyl transferase (TdT), which catalyzed polymerization of labeled nucleotides to free 3′-OH DNA ends in template-independent manner Fluorescein could be detected by anti-fluorescein antibody, conjugated with horse-radish-peroxidase (POD). After DAB reaction, stained cells were analyzed and counted under light microscope.

For Western blot, cells were harvested and lysed by lysis buffer. Western blot were performed with anti-PARP or anti-Pol(DNA polymerase) II antibody. During apoptosis, PARP is cleaved from a 113KD intact form into smaller 89KD and 24KD fragments.

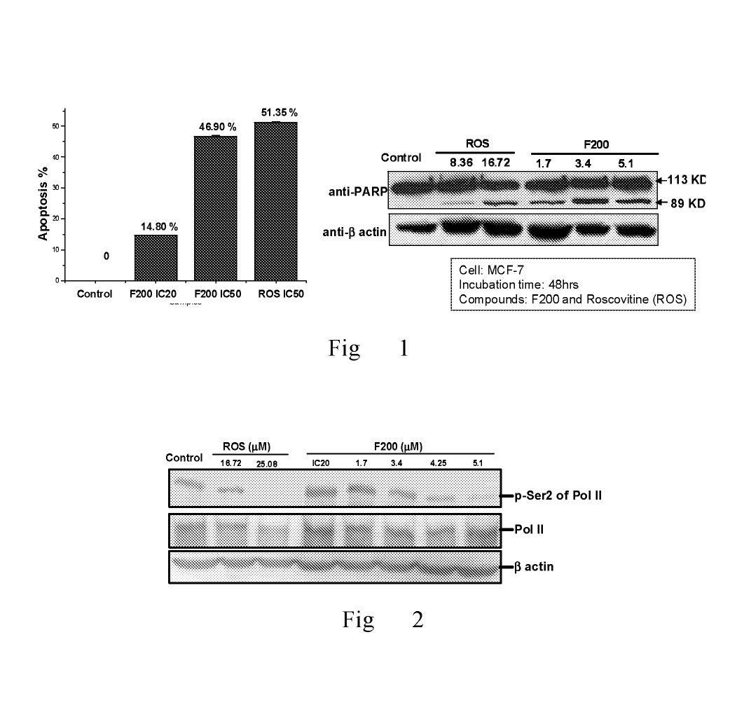

Results are shown in FIGS. 1 and 2.

It showed that compound 18 inhibited CDK9's activity on Pol II phosphorylation and induced apoptosis of cells.

### Example 3

### Acute Toxicity & Pharmacokinetic Determination

Preliminary toxicology study in mice: Mice were divided into four groups (n=10 in each), 500 mg/kg, 1,000 mg/kg or 2,000 mg/kg were administered respectively to three groups by gavage. Negative control is 0.5% CMC-Na. After two weeks, mice were sacrificed and tissue section of visceral organs were analyzed by Hematoxylin-Eosin staining

Results: LD50>2000 mg/kg (p.o.) MTD>500 mg/kg (p.o.)

These results demonstrated that Compound 18 has excellent tolerability in animals

TABLE II
PK profile of compound 18 (SG45 or F200): rat IV/PO study

| | AUC₍₀₋ₜ₎ | AUC_{(0-∞)} | MRT_{(0-∞)} | t_{1/2} | Tₘₐₓ | Vz | CL | Cₘₐₓ | |
|---|---|---|---|---|---|---|---|---|---|
| | μg/L * hr | μg/L * hr | hr | hr | hr | L/kg | L/hr/kg | ng/mL | F* % |
| IV (5 mg/kg) | 543.18 | 544.96 | 0.67 | 0.70 | 0.083 | 9.60 | 9.38 | 962.51 | |
| PO (20 mg/kg) | 1466.73 | 1486.74 | 1.67 | 1.24 | 0.58 | NA | NA | 895.39 | 67.51 |

This result showed that compound 18 has good oral availability and t 12 after oral administration.

### Example 4

### Inhibition of Tumor Cell Growth by Compound 18 (SG45 or F200)

Animal: SCID-bg mice
Tumor type: lung carcinoma (H460)
Treatment: Single PO dose for 21 days (starting at day 7 following tumor inoculation)
Endpoint: tumor volume and mean luciferase activity by Xenogen (Caliper) machine:
SCID-bg mice were implanted with NCI-H460 cells at 5×10⁶ cells/mouse on Day 0. Compound 18 treatment began on Day 7. Animals were administered orally once a day with 20 or 100 mg/kg compounds dissolved in vehicle (0.5% carboxymethylcellulose sodium) on Day 7 to Day 21. In model group, the vehicle was used instead of compound 18. Tumor volume and mean luciferase activity by Xenogen technology were measured once per there days.

TABLE III
Tumor growth inhibition

| | % Tumor Growth Inhibition at Day 21 |
|---|---|
| Compound 18 (20 mg/kg) | 41.3% |
| Compound 18 (150 mg/kg) | 56.5% |

It showed that compound 18 was able to inhibit tumor growth via apoptosis induction at 150 mg/kg after oral administration.

### Example 5

### Induction of Neutrophil Apoptosis by Compound 18 (SG45 or F200)

1. Acquire 5-10 ml blood from adult rabbit
2. Isolate neutrophils using RED BLOOD CELL LYSING BUFFER (SIGMA Cat No. R7757) and rabbit isolation kit (TBDsciences, Cat No. LZS11133)
3. Treat neutrophils with reference compounds or SG45 for 15 h
4. Apoptosis assay (Annexin-V and PI staining)

Harvest neutrophils by centrifugation at 1,000 rpm, 4° C. for 7 min Remove culture medium after centrifugation. Wash the cell pellet with pre-cooled PBS 200 l once. The re-suspend the cells with pre-cooled PBS 100 l. Incubate the cells with 2 l dye PI (BD Biosciences. Cat. No. 556463) and 2 l anti-Annexin V-PE (BD Biosciences. Cat. No. 556421) at room temperature under the darkness for 30 minutes. Remove the antibodies by centrifugation at 1,000 rpm, 4° C. for 2 min Resuspend the cells in 200 l pre-cooled PBS Finally, load the cell suspension to the FACS machine to acquire the staining results:

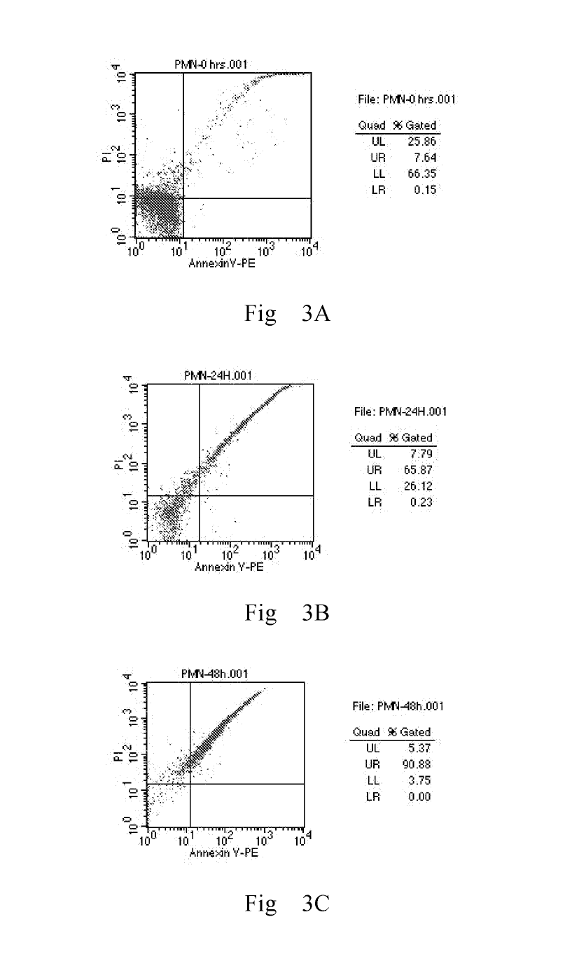

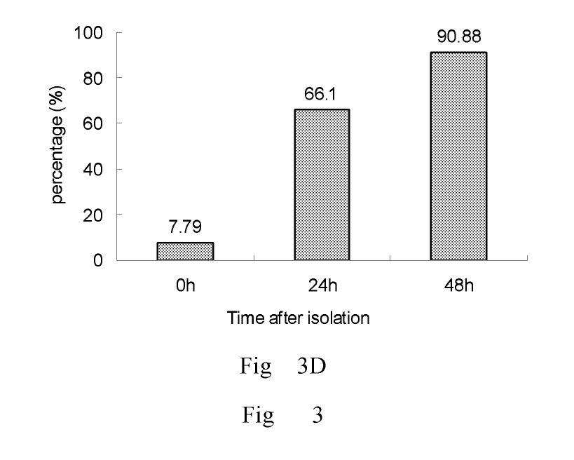

Results see FIG. 3.

It showed that healthy neutrophil quickly went into apoptosis in 48 h without any compound stimulation.

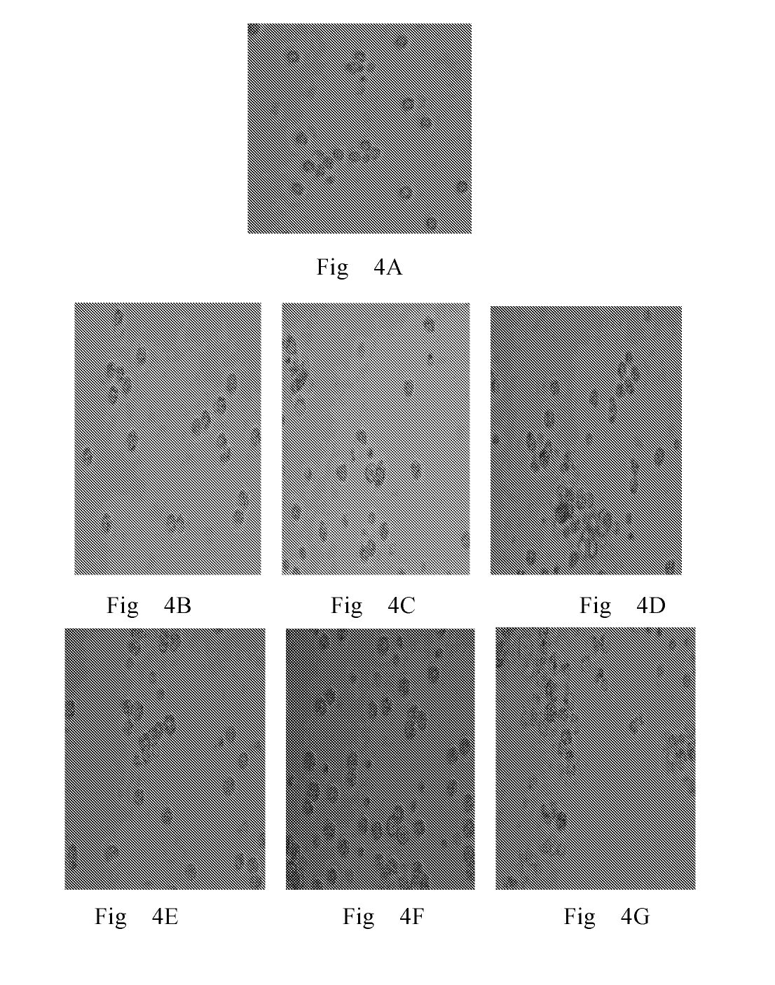

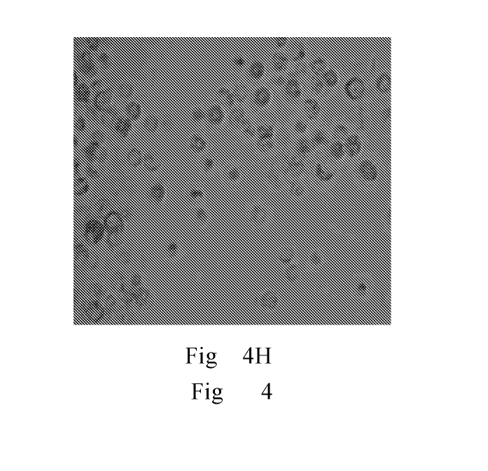

Results see FIG. 4.

It showed that with increasing dose of compound 18, typical apoptotic morphological changes, including blebbing, loss of cell membrane asymmetry and attachment, cell shrinkage etc. were observed.

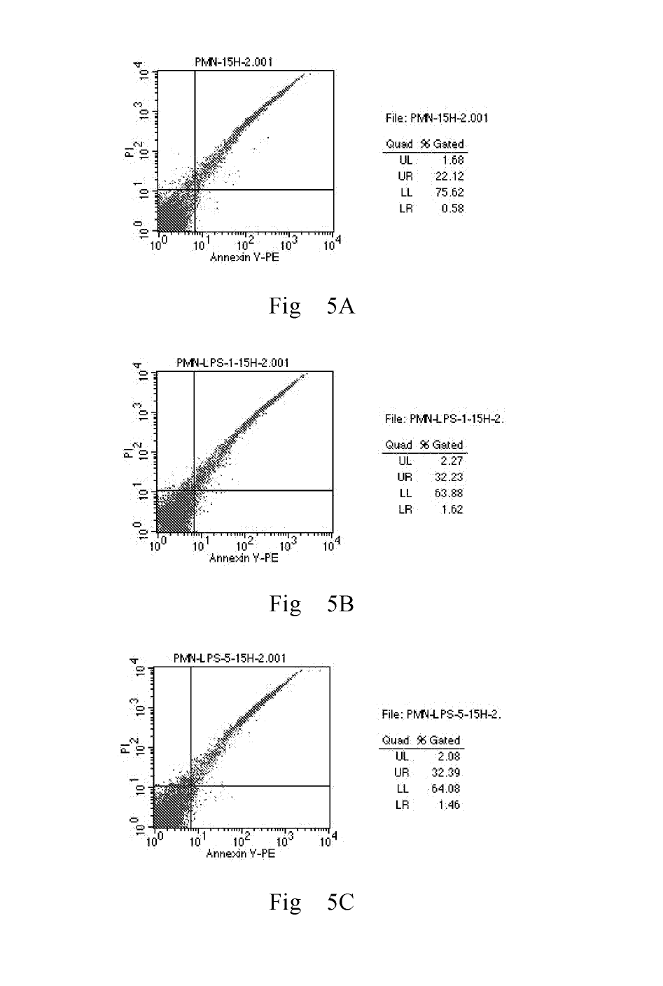

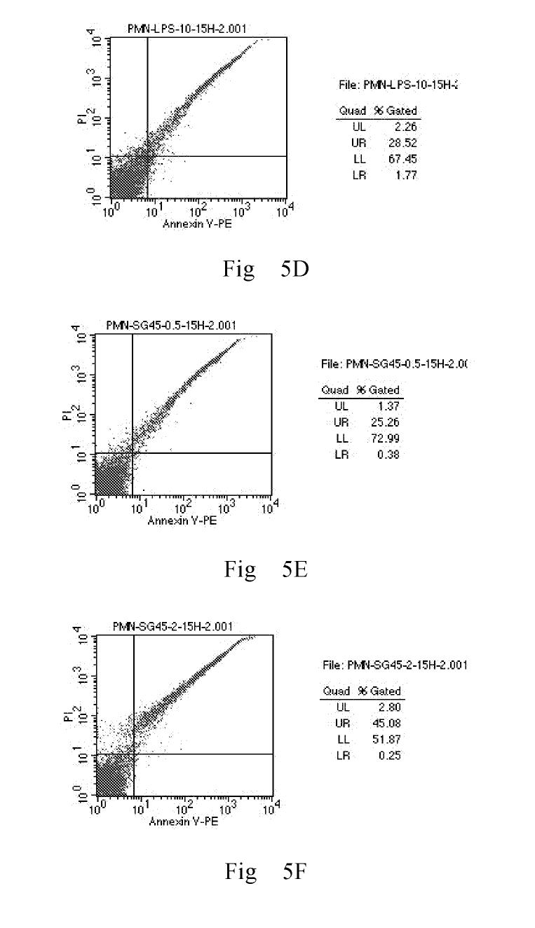

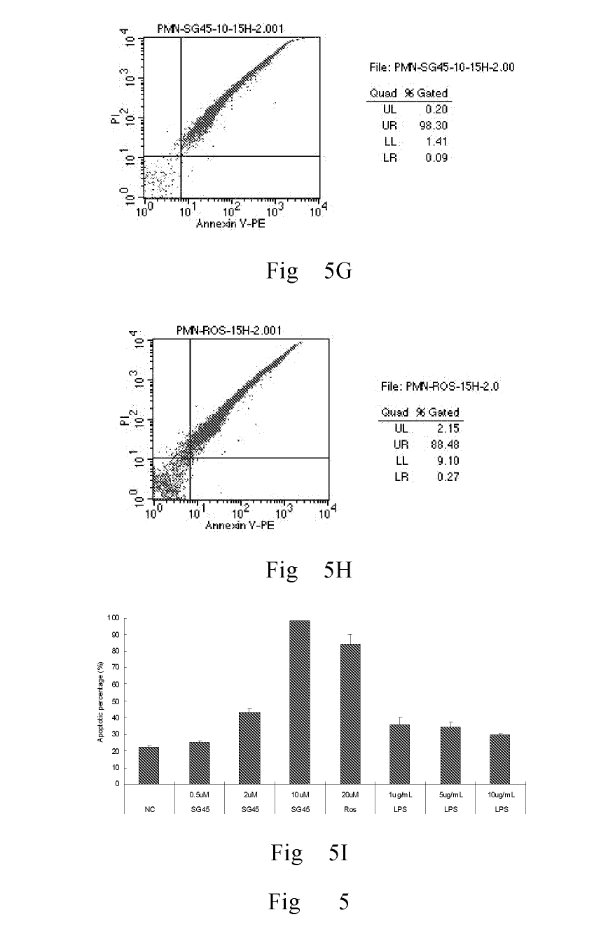

Results see FIG. 5.

Compound 18 showed stronger capability in inducing neutrophil apoptosis than Roscovitine in 15 h.

The examples and embodiments described herein are for illustrative purposes only and various modifications or changes suggested to persons skilled in the art are to be included within the spirit and purview of this application and scope of the appended claims.

1. Use of a compound, or pharmaceutically acceptable salt, solvate, or prodrug thereof, for preparing pharmaceutical agents of treating or preventing inflammatory disorder; the compound having the structure of Formula (Ia): 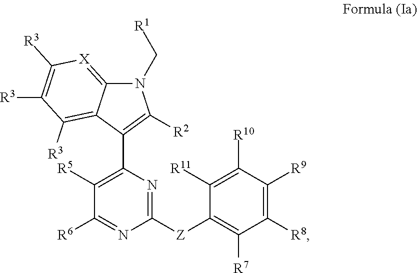
wherein:
X is —C(R³)— or —N—;
R¹ is unsubstituted C₁-C₆ alkyl, unsubstituted or substituted aryl, or unsubstituted or substituted heteroaryl;
R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —C(═O)O—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl.

2. The use of claim 1, wherein the inflammatory disorder is selected from disorders in which an increase of at least ten-fold in at least one of neutrophil number and eosinophil number is important for disease progress.

3. The use of claim 1, wherein:
X is N.

4. The use of claim 1, wherein:
X is —C(R³)—.

5. The use of claim 1, wherein:
Z is —NR^{a}—.

6. The use of claim 1, wherein:
R¹ is unsubstituted C₁-C₆ alkyl or unsubstituted or substituted phenyl;
each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃;
R², R⁵ and R⁶ are each H;
R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, or -L^{A}-L^{B}-R³²,
L^{A} is a covalent bond or an alkyl group;
L^{B} is a covalent bond, —O—, —NR³²—, —NH—, —CO(═O)—, —C(═O)NR³²—, —C(═O)NH—, —SO₂NR³²—, or —SO₂NH—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

7. The use of claim 1, wherein the compound having the structure of Formula (Ib): 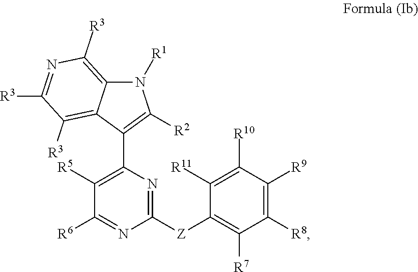
wherein:
R¹, R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂⁻, —CH₂CH₂—, —CH═CH—, —CH₂C(═O)NR^{a}—, —NR^{a}C(═O)CH₂—, —SO₂—, or —SO—;
R^{a} is H or alkyl.

8. The use of claim 7, wherein:
Z is —NR^{a}—.

9. The use of claim 8, wherein:
R¹ is unsubstituted C₁-C₆ alkyl;
each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃;
R², R⁵ and R⁶ are each H;
R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²,
L^{A} is a covalent bond or an alkyl group;
L^{B} is a covalent bond, —O—, —NR³²—, —NH—, —C(═O)O—, —C(═O)NR³²—, —C(═O)NH—, —SO₂NR³²—, or —SO₂NH—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

10. The use of claim 1, wherein the compound having the structure of Formula (Ic): 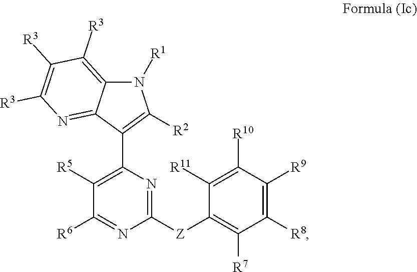
wherein:
R¹, R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl.

11. The use of claim 10, wherein:
Z is —NR^{a}—.

12. The use of claim 10, wherein:
R¹ is unsubstituted C₁-C₆ alkyl;
each R³ is independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, and —CH₃;
R², R⁵ and R⁶ are each H;
R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²,
L^{A} is a covalent bond or an alkyl group;
L^{B} is a covalent bond, —O—, —NR³²—, —NH—, —C(═O)O—, —C(═O)NR³²—, —C(═O)NH—, —SO₂NR³²—, or —SO₂NH—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃.

13. The use of claim 1, wherein the compound selected from:
3-(4-(1-Methyl-1H-indol-3-yl)pyrimidin-2-ylamino)phenol (Compound 1);
4-(4-(1-Methyl-1H-indol-3-yl)pyrimidin-2-ylamino)phenol (Compound 2);
3-Chloro-4-(4-(1-methyl-1H-indol-3-yl)pyrimidin-2-ylamino)phenol (Compound 3);
3-Methyl-4-[4-(1-methyl-1H-indol-3-yl)-pyrimidin-2-ylamino]-phenol (Compound 4);
3,5-Dimethyl-4-[4-(1-methyl-1H-indol-3-yl)-pyrimidin-2-ylamino]-phenol (Compound 5);
4-[4-(1-Ethyl-1H-indol-3-yl)-pyrimidin-2-ylamino]-phenol (Compound 6);
3-[4-(1-Ethyl-1H-indol-3-yl)-pyrimidin-2-ylamino]-phenol (Compound 7);
4-[4-(1-Ethyl-1H-indol-3-yl)-pyrimidin-2-ylamino]-3-methyl-phenol (Compound 8);
4-[4-(1-Ethyl-1H-indol-3-yl)-pyrimidin-2-ylamino]-3,5-dimethyl-phenol (Compound 9);
4-[4-(1-Benzyl-1H-indol-3-yl)-pyrimidin-2-ylamino]-phenol (Compound 10);
3-[4-(1-Benzyl-1H-indol-3-yl)-pyrimidin-2-ylamino]-phenol (Compound 11);
4-[4-(1-Ethyl-1H-pyrrolo[2,3-b]pyridin-3-yl)-pyrimidin-2-ylamino]-phenol (Compound 12);
3-[4-(1-Ethyl-1H-pyrrolo[2,3-b]pyridin-3-yl)-pyrimidin-2-ylamino]-phenol (Compound 13);
[4-(1-Ethyl-1H-pyrrolo[2,3-b]pyridin-3-yl)-pyrimidin-2-yl]-(3-nitro-phenyl)-amine (Compound 14);
4-[4-(1-Propyl-1H-pyrrolo[2,3-b]pyridin-3-yl)-pyrimidin-2-ylamino]-phenol (Compound 15);
3-[4-(1-Propyl-1H-pyrrolo[2,3-b]pyridin-3-yl)-pyrimidin-2-ylamino]-phenol (Compound 16);
(3-Nitro-phenyl)-[4-(1-propyl-1H-pyrrolo[2,3-b]pyridin-3-yl)-pyrimidin-2-yl]-amine (Compound 17);
3-(4-(1-Ethyl-1H-pyrrolo[2,3-b]pyridin-3-yl)pyrimidin-2-ylamino)benzenesulfonamide (Compound 18);
3-(4-(1-Ethyl-1H-pyrrolo[2,3-b]pyridin-3-yl)pyrimidin-2-ylamino)benzamide (Compound 19);
3-(4-(1-ethyl-1H-pyrrolo[2,3-b]pyridin-3-yl)pyrimidin-2-ylamino)benzonitrile (Compound 20);
4-(4-(1-ethyl-1H-pyrrolo[2,3-b]pyridin-3-yl)pyrimidin-2-ylamino)benzenesulfonamide (Compound 21);
3-(4-(1-propyl-1H-pyrrolo[2,3-b]pyridin-3-yl)pyrimidin-2-ylamino)benzenesulfonamide (Compound 22);
4-(4-(1-Propyl-1H-pyrrolo[2,3-b]pyridin-3-yl)pyrimidin-2-ylamino)benzenesulfonamide (Compound 23);
N-(4-Methyl-3-(morpholinosulfonyl)phenyl)-4-(1-propyl-1H-pyrrolo[2,3-b]pyridin-3-yl)pyrimidin-2-amine (Compound 24);
N-(3-(Piperazin-1-ylsulfonyl)phenyl)-4-(1-propyl-1H-pyrrolo[2,3-b]pyridin-3-yl)pyrimidin-2-amine (Compound 25);
N-(4-(Piperazin-1-ylsulfonyl)phenyl)-4-(1-propyl-1H-pyrrolo[2,3-b]pyridin-3-yl)pyrimidin-2-amine (Compound 26);
N-(4-(Morpholinosulfonyl)phenyl)-4-(1-propyl-1H-pyrrolo[2,3-b]pyridin-3-yl)pyrimidin-2-amine (Compound 27);
N-(3-(4-Methylpiperazin-1-ylsulfonyl)phenyl)-4-(1-propyl-1H-pyrrolo[2,3-b]pyridin-3-yl)pyrimidin-2-amine (Compound 28);
N-(4-(4-Methylpiperazin-1-ylsulfonyl)phenyl)-4-(1-propyl-1H-pyrrolo[2,3-b]pyridin-3-yl)pyrimidin-2-amine (Compound 29);
N-(3-Morpholinophenyl)-4-(1-propyl-1H-pyrrolo[2,3-b]pyridin-3-yl)pyrimidin-2-amine (Compound 30);
N-(4-Morpholinophenyl)-4-(1-propyl-1H-pyrrolo[2,3-b]pyridin-3-yl)pyrimidin-2-amine (Compound 31);
N-(3-(Piperazin-1-yl)phenyl)-4-(1-propyl-1H-pyrrolo[2,3-b]pyridin-3-yl)pyrimidin-2-amine (Compound 32);
N-(4-(Piperazin-1-yl)phenyl)-4-(1-propyl-1H-pyrrolo[2,3-b]pyridin-3-yl)pyrimidin-2-amine (Compound 33);
(4-Methylpiperazin-1-yl)(3-(4-(1-propyl-1H-pyrrolo[2,3-b]pyridin-3-yl)pyrimidin-2-ylamino)phenyl)methanone (Compound 34);
N-(4-(4-Methylpiperazin-1-yl)phenyl)-4-(1-propyl-1H-pyrrolo[2,3-b]pyridin-3-yl)pyrimidin-2-amine (Compound 35);
1-(4-(3-(4-(1-Propyl-1H-pyrrolo[2,3-b]pyridin-3-yl)pyrimidin-2-ylamino)phenyl)piperazin-1-yl)ethanone (Compound 36);
1-(4-(4-(4-(1-Propyl-1H-pyrrolo[2,3-b]pyridin-3-yl)pyrimidin-2-ylamino)phenyl)piperazin-1-yl)ethanone (Compound 37);
4-(4-(5-Chloro-7-methyl-1-propyl-1H-pyrrolo[3,2-b]pyridin-3-yl)pyrimidin-2-ylamino)phenol (Compound 38);
4-(4-(5-Chloro-7-methyl-1-propyl-1H-pyrrolo[3,2-b]pyridin-3-yl)pyrimidin-2-ylamino)benzenesulfonamide (Compound 39);
3-(4-(5-Chloro-7-methyl-1-propyl-1H-pyrrolo[3,2-b]pyridin-3-yl)pyrimidin-2-ylamino)phenol (Compound 40);
4-(4-(5-Chloro-7-methyl-1-propyl-1H-pyrrolo[3,2-b]pyridin-3-yl)pyrimidin-2-ylamino)-3-methylphenol (Compound 41);
3-(4-(5-Chloro-7-methyl-1-propyl-1H-pyrrolo[3,2-b]pyridin-3-yl)pyrimidin-2-ylamino)benzenesulfonamide (Compound 42);
4-(5-Chloro-7-methyl-1-propyl-1H-pyrrolo[3,2-b]pyridin-3-yl)-N-(4-methyl-3-(morpholinosulfonyl)phenyl)pyrimidin-2-amine (Compound 43);
4-(5-Chloro-7-methyl-1-propyl-1H-pyrrolo[3,2-b]pyridin-3-yl)-N-(3-(piperazin-1-ylsulfonyl)phenyl)pyrimidin-2-amine (Compound 44);
4-(5-Chloro-7-methyl-1-propyl-1H-pyrrolo[3,2-b]pyridin-3-yl)-N-(4-(piperazin-1-ylsulfonyl)phenyl)pyrimidin-2-amine (Compound 45);
4-(5-Chloro-7-methyl-1-propyl-1H-pyrrolo[3,2-b]pyridin-3-yl)-N-(3-(piperazin-1-yl)phenyl)pyrimidin-2-amine (Compound 46);
4-(5-Chloro-7-methyl-1-propyl-1H-pyrrolo[3,2-b]pyridin-3-yl)-N-(4-(piperazin-1-yl)phenyl)pyrimidin-2-amine (Compound 47);
4-(5-Chloro-7-methyl-1-propyl-1H-pyrrolo[3,2-b]pyridin-3-yl)-N-(3-morpholinophenyl)pyrimidin-2-amine (Compound 48);
4-(5-Chloro-7-methyl-1-propyl-1H-pyrrolo[3,2-b]pyridin-3-yl)-N-(4-morpholinophenyl)pyrimidin-2-amine (Compound 49);
1-(4-(3-(4-(5-Chloro-7-methyl-1-propyl-1H-pyrrolo[3,2-b]pyridin-3-yl)pyrimidin-2-ylamino)phenyl)piperazin-1-yl)ethanone (Compound 50);
1-(4-(4-(4-(5-Chloro-7-methyl-1-propyl-1H-pyrrolo[3,2-b]pyridin-3-yl)pyrimidin-2-ylamino)phenyl)piperazin-1-yl)ethanone (Compound 51);
(3-(4-(5-Chloro-7-methyl-1-propyl-1H-pyrrolo[3,2-b]pyridin-3-yl)pyrimidin-2-ylamino)phenyl)(morpholino)methanone (Compound 52);
(4-(4-(5-Chloro-7-methyl-1-propyl-1H-pyrrolo[3,2-b]pyridin-3-yl)pyrimidin-2-ylamino)phenyl)(morpholino)methanone (Compound 53);
3-(4-(7-Methyl-1-propyl-1H-pyrrolo[3,2-b]pyridin-3-yl)pyrimidin-2-ylamino)benzenesulfonamide (Compound 54);
4-(4-(7-Methyl-1-propyl-1H-pyrrolo[3,2-b]pyridin-3-yl)pyrimidin-2-ylamino)benzenesulfonamide (Compound 55);
4-(7-Methyl-1-propyl-1H-pyrrolo[3,2-b]pyridin-3-yl)-N-(3-(piperazin-1-ylsulfonyl)phenyl)pyrimidin-2-amine (Compound 56);
4-(7-Methyl-1-propyl-1H-pyrrolo[3,2-b]pyridin-3-yl)-N-(4-(piperazin-1-ylsulfonyl)phenyl)pyrimidin-2-amine (Compound 57);
4-(7-Methyl-1-propyl-1H-pyrrolo[3,2-b]pyridin-3-yl)-N-(3-(piperazin-1-yl)phenyl)pyrimidin-2-amine (Compound 58);
4-(7-Methyl-1-propyl-1H-pyrrolo[3,2-b]pyridin-3-yl)-N-(4-(piperazin-1-yl)phenyl)pyrimidin-2-amine (Compound 59);
4-(7-Methyl-1-propyl-1H-pyrrolo[3,2-b]pyridin-3-yl)-N-(3-morpholinophenyl)pyrimidin-2-amine (Compound 60);
4-(7-Methyl-1-propyl-1H-pyrrolo[3,2-b]pyridin-3-yl)-N-(4-morpholinophenyl)pyrimidin-2-amine (Compound 61);
1-(4-(3-(4-(7-Methyl-1-propyl-1H-pyrrolo[3,2-b]pyridin-3-yl)pyrimidin-2-ylamino)phenyl)piperazin-1-yl)ethanone (Compound 62);
1-(4-(4-(4-(7-Methyl-1-propyl-1H-pyrrolo[3,2-b]pyridin-3-yl)pyrimidin-2-ylamino)phenyl)piperazin-1-yl)ethanone (Compound 63);
3-(4-(1-Ethyl-7-methyl-1H-pyrrolo[3,2-b]pyridin-3-yl)pyrimidin-2-ylamino)benzenesulfonamide (Compound 64);
4-(4-(1-Ethyl-7-methyl-1H-pyrrolo[3,2-b]pyridin-3-yl)pyrimidin-2-ylamino)benzenesulfonamide (Compound 65);
(3-(4-(1-Ethyl-7-methyl-1H-pyrrolo[3,2-b]pyridin-3-yl)pyrimidin-2-ylamino)phenyl)(morpholino)methanone (Compound 66);
(4-(4-(1-Ethyl-7-methyl-1H-pyrrolo[3,2-b]pyridin-3-yl)pyrimidin-2-ylamino)phenyl)(morpholino)methanone (Compound 67);
4-(1-Ethyl-7-methyl-1H-pyrrolo[3,2-b]pyridin-3-yl)-N-(3-(piperazin-1-yl)phenyl)pyrimidin-2-amine (Compound 68);
4-(1-Ethyl-7-methyl-1H-pyrrolo[3,2-b]pyridin-3-yl)-N-(4-(piperazin-1-yl)phenyl)pyrimidin-2-amine (Compound 69);
4-(4-(7-Chloro-1-ethyl-1H-pyrrolo[2,3-c]pyridin-3-yl)pyrimidin-2-ylamino)phenol (Compound 70);
4-(4-(7-Chloro-1-ethyl-1H-pyrrolo[2,3-c]pyridin-3-yl)pyrimidin-2-ylamino)benzenesulfonamide (Compound 71);
3-(4-(7-Chloro-1-ethyl-1H-pyrrolo[2,3-c]pyridin-3-yl)pyrimidin-2-ylamino)phenol (Compound 72);
3-(4-(7-Chloro-1-ethyl-1H-pyrrolo[2,3-c]pyridin-3-yl)pyrimidin-2-ylamino)benzenesulfonamide (Compound 73);
4-(7-Chloro-1-ethyl-1H-pyrrolo[2,3-c]pyridin-3-yl)-N-(4-methyl-3-(morpholinosulfonyl)phenyl)pyrimidin-2-amine (Compound 74);
(3-(4-(7-Chloro-1-ethyl-1H-pyrrolo[2,3-c]pyridin-3-yl)pyrimidin-2-ylamino)phenyl)(morpholino)methanone (Compound 75);
(4-(4-(7-Chloro-1-ethyl-1H-pyrrolo[2,3-c]pyridin-3-yl)pyrimidin-2-ylamino)phenyl)(morpholino)methanone (Compound 76);
4-(7-Chloro-1-ethyl-1H-pyrrolo[2,3-c]pyridin-3-yl)-N-(3-(piperazin-1-ylsulfonyl)phenyl)pyrimidin-2-amine (Compound 77);
4-(7-Chloro-1-ethyl-1H-pyrrolo[2,3-c]pyridin-3-yl)-N-(4-(piperazin-1-ylsulfonyl)phenyl)pyrimidin-2-amine (Compound 78);
4-(7-Chloro-1-ethyl-1H-pyrrolo[2,3-c]pyridin-3-yl)-N-(3-(piperazin-1-yl)phenyl)pyrimidin-2-amine (Compound 79);
4-(7-Chloro-1-ethyl-1H-pyrrolo[2,3-c]pyridin-3-yl)-N-(4-(piperazin-1-yl)phenyl)pyrimidin-2-amine (Compound 80);
4-(7-Chloro-1-ethyl-1H-pyrrolo[2,3-c]pyridin-3-yl)-N-(3-morpholinophenyl)pyrimidin-2-amine (Compound 81);
4-(7-Chloro-1-ethyl-1H-pyrrolo[2,3-c]pyridin-3-yl)-N-(4-morpholinophenyl)pyrimidin-2-amine (Compound 82);
3-(4-(1-Ethyl-1H-pyrrolo[2,3-c]pyridin-3-yl)pyrimidin-2-ylamino)phenol (Compound 83);
3-(4-(1-Ethyl-1H-pyrrolo[2,3-c]pyridin-3-yl)pyrimidin-2-ylamino)benzenesulfonamide (Compound 84);
4-(4-(1-Ethyl-1H-pyrrolo[2,3-c]pyridin-3-yl)pyrimidin-2-ylamino)phenol (Compound 85);
4-(4-(1-Ethyl-1H-pyrrolo[2,3-c]pyridin-3-yl)pyrimidin-2-ylamino)benzenesulfonamide (Compound 86);
4-(1-Ethyl-1H-pyrrolo[2,3-c]pyridin-3-yl)-N-(4-methyl-3-(morpholinosulfonyl)phenyl)pyrimidin-2-amine (Compound 87);
(3-(4-(1-Ethyl-1H-pyrrolo[2,3-c]pyridin-3-yl)pyrimidin-2-ylamino)phenyl)(morpholino)methanone (Compound 88);
(4-(4-(1-Ethyl-1H-pyrrolo[2,3-c]pyridin-3-yl)pyrimidin-2-ylamino)phenyl)(morpholino)methanone (Compound 89);
1-(4-(3-(4-(1-Ethyl-1H-pyrrolo[2,3-c]pyridin-3-yl)pyrimidin-2-ylamino)benzoyl)piperazin-1-yl)ethanone (Compound 90);
1-(4-(4-(4-(1-Ethyl-1H-pyrrolo[2,3-c]pyridin-3-yl)pyrimidin-2-ylamino)benzoyl)piperazin-1-yl)ethanone (Compound 91);
4-(1-Ethyl-1H-pyrrolo[2,3-c]pyridin-3-yl)-N-(3-(piperazin-1-yl)phenyl)pyrimidin-2-amine (Compound 92);
4-(1-Ethyl-1H-pyrrolo[2,3-c]pyridin-3-yl)-N-(4-(piperazin-1-yl)phenyl)pyrimidin-2-amine (Compound 93);
4-(1-Ethyl-1H-pyrrolo[2,3-c]pyridin-3-yl)-N-(3-morpholinophenyl)pyrimidin-2-amine (Compound 94);
4-(1-Ethyl-1H-pyrrolo[2,3-c]pyridin-3-yl)-N-(4-morpholinophenyl)pyrimidin-2-amine (Compound 95);
4-(4-(7-Chloro-1-propyl-1H-pyrrolo[2,3-c]pyridin-3-yl)pyrimidin-2-ylamino)benzenesulfonamide (Compound 96);
3-(4-(7-Chloro-1-propyl-1H-pyrrolo[2,3-c]pyridin-3-yl)pyrimidin-2-ylamino)benzenesulfonamide (Compound 97);
1-(4-(3-(4-(7-Chloro-1-propyl-1H-pyrrolo[2,3-c]pyridin-3-yl)pyrimidin-2-ylamino)benzoyl)piperazin-1-yl)ethanone (Compound 98);
1-(4-(4-(4-(7-Chloro-1-propyl-1H-pyrrolo[2,3-c]pyridin-3-yl)pyrimidin-2-ylamino)benzoyl)piperazin-1-yl)ethanone (Compound 99);
4-(7-Chloro-1-propyl-1H-pyrrolo[2,3-c]pyridin-3-yl)-N-(4-morpholinophenyl)pyrimidin-2-amine (Compound 100);
4-(7-Chloro-1-propyl-1H-pyrrolo[2,3-c]pyridin-3-yl)-N-(4-methyl-3-(morpholinosulfonyl)phenyl)pyrimidin-2-amine (Compound 101);
N-(4-Methyl-3-(morpholinosulfonyl)phenyl)-4-(1-propyl-1H-pyrrolo[2,3-c]pyridin-3-yl)pyrimidin-2-amine (Compound 102);
N-(4-Morpholinophenyl)-4-(1-propyl-1H-pyrrolo[2,3-c]pyridin-3-yl)pyrimidin-2-amine (Compound 103);
N-(3-(Piperazin-1-yl)phenyl)-4-(1-propyl-1H-pyrrolo[2,3-c]pyridin-3-yl)pyrimidin-2-amine (Compound 104);
N-(4-(Piperazin-1-yl)phenyl)-4-(1-propyl-1H-pyrrolo[2,3-c]pyridin-3-yl)pyrimidin-2-amine (Compound 105);
1-(4-(4-(4-(1-Propyl-1H-pyrrolo[2,3-c]pyridin-3-yl)pyrimidin-2-ylamino)benzoyl)piperazin-1-yl)ethanone (Compound 106);
Piperazin-1-yl(3-(4-(1-propyl-1H-pyrrolo[2,3-c]pyridin-3-yl)pyrimidin-2-ylamino)phenyl)methanone (Compound 107);
Morpholino(3-(4-(1-propyl-1H-pyrrolo[2,3-c]pyridin-3-yl)pyrimidin-2-ylamino)phenyl)methanone (Compound 108);
Piperazin-1-yl(4-(4-(1-propyl-1H-pyrrolo[2,3-c]pyridin-3-yl)pyrimidin-2-ylamino)phenyl)methanone (Compound 109);
Morpholino(4-(4-(1-propyl-1H-pyrrolo[2,3-c]pyridin-3-yl)pyrimidin-2-ylamino)phenyl)methanone (Compound 110);
3-(4-(1-Propyl-1H-pyrrolo[2,3-c]pyridin-3-yl)pyrimidin-2-ylamino)benzenesulfonamide (Compound 111);
4-(4-(1-Propyl-1H-pyrrolo[2,3-c]pyridin-3-yl)pyrimidin-2-ylamino)benzenesulfonamide (Compound 112).

14. A method for treating or preventing inflammatory disorder in a patient, comprising administrating to the patient an effective amount of a compound, or pharmaceutically acceptable salt, solvate, or prodrug thereof, the compound having the structure of Formula (Ia): 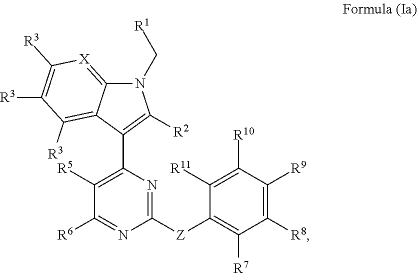
wherein:
X is —C(R³)— or —N—;
R¹ is unsubstituted C₁-C₆ alkyl, unsubstituted or substituted aryl, or unsubstituted or substituted heteroaryl;
R², R³, R⁵, R⁶, R⁷, R⁸, R⁹, R¹⁰ and R¹¹ are each independently selected from the group consisting of H, halogen, —NH₂, —NO₂, —CN, —OH, —CO₂H, —CONH₂, —SO₃H, —SO₂NH₂, —SO₂CH₃, —OCH₃, —CF₃, —CH₃, and -L^{A}-L^{B}-R³²;
L^{A} is a covalent bond or an alkyl group;
L^{B} is a covalent bond, —O—, —NR³²—, —NH—, —C(═O)—, —CO(═O)—, —OC(═O)—, —C(═O)NR³²—, —C(═O)NH—, —NR³²C(═O)—, —NHC(═O)—, —SO₂—, —SO₂NR³²—, —NR³²SO₂—, —SO₂NH—, or —NHSO₂—;
R³² is H, substituted or unsubstituted alkyl, haloalkyl, substituted or unsubstituted aryl, substituted or unsubstituted heteroaryl, substituted or unsubstituted non-aromatic heterocycle, or substituted or unsubstituted cycloalkyl; wherein any R³² group, when substituted, is substituted with one or more groups selected from halogen, —NH₂, —NO₂, —CN, —OH, —CO₂H, —CONH₂, —SO₃H, —SO₂NH₂, —SO₂CH₃, —OCH₃, and —CF₃;
Z is —NR^{a}C(═O)—, —C(═O)NR^{a}—, —NR^{a}SO₂—, —SO₂NR^{a}—, —NR^{a}—, —CH₂NR^{a}—, —NR^{a}CH₂—, —CH₂—, —CH₂CH₂—, —CH═CH—, —CH₂C(═O)NR^{a}—, —NR^{a}C(═O)CH₂—, —SO₂—, or —SO—;
R^{a} is H or alkyl.

15. The method of claim 14, wherein the inflammatory disorder is selected from disorders in which an increase of at least 10-fold in at least one of neutrophil number and eosinophil number is important for disease progress.

16. The use of claim 2, wherein the inflammatory disorder is selected from the group consisting of Acute Exacerbation of Chronic Obstructive Pulmonary Disease (COPD), Acute Respiratory Distress Syndrome (ARDS), Sepsis, and Rheumatoid Arthritis.

17. The method of claim 15, wherein the inflammatory disorder is selected from the group consisting of Acute Exacerbation of Chronic Obstructive Pulmonary Disease (COPD), Acute Respiratory Distress Syndrome (ARDS), Sepsis, and Rheumatoid Arthritis.
